(19) United States
(12) Patent Application Publication
(10) Pub. No.: US 2009/0270407 A1
(43) Pub. Date: Oct. 29, 2009

(54) FUNGICIDAL ISOXAZOLIDINES

(75) Inventors: CHI-PING TSENG, Wilmington, DE (US); Brenton Todd Smith, Exton, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/428,611

(22) Filed: Apr. 23, 2009

Related U.S. Application Data
(60) Provisional application No. 61/125,624, filed on Apr. 25, 2008.

(51) Int. Cl. A01N 43/58; C07D 413/04; C07D 417/08; A01N 43/60; A01N 43/54; A01N 43/40; A01P 3/00

(52) U.S. Cl. 514/252.05; 546/272.1; 544/405; 544/333; 544/238; 546/270.1; 514/255.05; 514/256; 514/340; 514/338

(57) ABSTRACT

Disclosed are compounds of Formula 1, including all geometric and stereoisomers, N-oxides, and salts thereof,

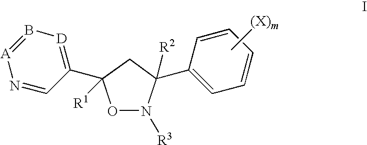

wherein
A, B, D, R¹, R², R³, X and m are as defined in the disclosure.

Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain fungicidal isoxazolidines their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

U.S. Pat. No. 6,313,147 discloses isoxazolidine derivatives of Formula i

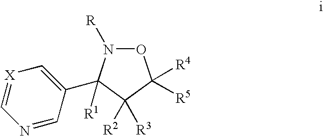

and their use as fungicides.

### SUMMARY OF THE INVENTION

This invention is directed to a compound of Formula 1 (including all geometric and stereoisomers), N-oxides, and salts thereof, and compositions containing them and their use for controlling fungal diseases in plants:

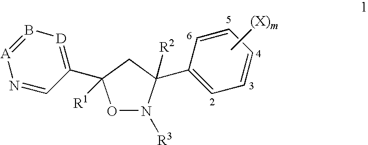

wherein
each A, B and D is independently N or CH,
provided that no more than one of A, B and D is N;
R¹ is H, C₁-C₃ alkyl, C₁-C₃ haloalkyl, C₃-C₅ cycloalkyl, C₄-C₆ cycloalkylalkyl, C₄-C₆ alkylcycloalkyl or C₃-C₅ halocycloalkyl;
R² is H, C₁-C₆ alkyl, C₃-C₅ cycloalkyl, C₄-C₆ alkylcycloalkyl, C₄-C₆ cycloalkylalkyl or C₄-C₆ halocycloalkylalkyl;
R³ is C₁-C₁₂ alkyl, —CH₂(C₁-C₁₁ haloalkyl), —CH₂(C₁-C₁₁ alkoxyalkyl), C₃-C₇ cycloalkyl, C₃-C₇ halocycloalkyl or C₄-C₇ cycloalkylalkyl, each optionally substituted with one or more substituents selected from the group consisting of CN, C₁-C₆ alkyl, C₁-C₆ haloalkyl, C₃-C₈ cycloalkyl, C₃-C₈ halocycloalkyl or G¹;
each X is independently halogen, cyano, nitro, C₁-C₆ alkyl, C₃-C₈ cycloalkyl, C₄-C₁₀ alkylcycloalkyl, C₄-C₁₀ cycloalkylalkyl, C₅-C₁₂ alkylcycloalkylalkyl, C₂-C₇ cyanoalkyl, C₂-C₈ alkoxyhaloalkyl, C₁-C₆ alkoxy, C₃-C₈ cycloalkoxy, C₄-C₁₀ cycloalkylalkoxy, C₁-C₆ alkylthio, C₃-C₈ cycloalkylthio, C₁-C₆ alkylsulfinyl, C₁-C₆ alkylsulfonyl, C₃-C₈ cycloalkylsulfonyl, C₃-C₈ cycloalkylsulfinyl, C₂-C₈ dialkylaminosulfonyl, C₂-C₈ alkoxycarbonyl, C₂-C₈ dialkylamino, C₄-C₁₂ dialkylimido, C₃-C₁₀ trialkylsilyl or G²; or
a pair of X substituents attached to contiguous carbon atoms are taken together with said carbon atoms to form a fused phenyl ring, a fused 5- to 6-membered nonaromatic carbocyclic ring, a fused 5- or 6-membered heteroaromatic ring or a fused 5- to 6-membered nonaromatic heterocyclic ring, each fused ring optionally substituted with up to 4 substituents independently selected from R⁴ on carbon ring members and R⁵ on nitrogen ring members;
each G¹ and G² is independently phenyl, benzyl, phenoxy, phenylsulfonyl, pyridinylmethyl, a 3- to 7-membered nonaromatic carbocyclic or heterocyclic ring, a 5- to 6-membered heteroaromatic ring or an 8- to 10-membered aromatic carbobicyclic or heterobicyclic ring system, each optionally substituted with up to 4 substituents independently selected from R⁴ on carbon ring members and R⁵ on nitrogen ring members
each R⁴ is halogen, cyano, nitro, C₁-C₆ alkyl, C₁-C₆ haloalkyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₂ alkylcycloalkylalkyl, C₂-C₈ alkoxyalkyl, C₂-C₈ haloalkoxyalkyl, C₂-C₈ alkoxyhaloalkyl, C₂-C₈ haloalkoxyhaloalkyl, C₄-C₁₀ cycloalkoxyalkyl, C₄-C₁₀ halocycloalkoxyalkyl, C₃-C₁₀ alkoxyalkoxyalkyl, C₁-C₆ alkoxy, C₁-C₆ haloalkoxy, C₃-C₈ cycloalkoxy, C₃-C₈ halocycloalkoxy, C₄-C₁₀ cycloalkylalkoxy, C₂-C₈ alkoxyalkoxy, C₂-C₈ haloalkoxyalkoxy, C₂-C₈ alkoxyhaloalkoxy, C₂-C₈ haloalkoxyhaloalkoxy, C₃-C₁₀ alkoxycarbonylalkoxy, C₂-C₈ alkylthioalkyl, C₁-C₆ alkylthio, C₁-C₆ haloalkylthio, C₃-C₈ cycloalkylthio, C₂-C₈ (alkylcarbonyl)thio, C₂-C₈ (alkylthio)carbonyl, C₂-C₈ alkoxycarbonyl, C₄-C₁₀ cycloalkoxycarbonyl, C₅-C₁₂ cycloalkylalkoxycarbonyl, C₃₋C₁₀ dialkylaminocarbonyl, C₂-C₈ alkylsulfinylalkyl, C₂-C₈ alkylsulfonylalkyl, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃₋C₈ cycloalkylsulfinyl, C₂-C₈ dialkylaminosulfonyl, C₃-C₁₀ dialkylaminoalkyl, C₂-C₈ dialkylamino, C₃-C₁₀ dialkylamidino, C₂-C₇ cyanoalkyl, C₃-C₁₀ trialkylsilyl, C₃-C₁₀ halotrialkylsilyl, phenyl, pyridinyl, thienyl, naphthalenyl, phenoxy, phenylsulfonyl, pyridinylmethyl or benzyl;
each R⁵ is C₁-C₆ alkyl, C₁-C₆ haloalkyl, C₃-C₈ cycloalkyl, C₃-C₈ halocycloalkyl, C₄-C₁₀ cycloalkylalkyl, C₄-C₁₀ halocycloalkylalkyl, C₄-C₁₀ alkylcycloalkyl, C₅-C₁₂ alkylcycloalkylalkyl, C₆-C₁₄ cycloalkylcycloalkyl, C₄-C₁₀ cycloalkoxyalkyl, C₄-C₁₀ halocycloalkoxyalkyl, C₂-C₈ alkoxyalkyl, C₂-C₈ haloalkoxyalkyl, C₃-C₁₀ alkoxyalkoxyalkyl, C₂-C₈ alkoxycarbonyl, C₄-C₁₀ cycloalkoxycarbonyl, C₅-C₁₂ cycloalkylalkoxycarbonyl, C₃-C₁₀ dialkylaminocarbonyl, C₂-C₈ (alkylthio)carbonyl, C₁-C₆ alkylthio, C₁-C₆ haloalkylthio, C₃-C₈ cycloalkylthio, C₂-C₈ alkylthioalkyl, C₂-C₈ alkylsulfinylalkyl, C₂-C₈ alkylsulfonylalkyl, C₁-C₆ haloalkylsulfonyl, C₃-C₈ cycloalkylsulfonyl, C₂-C₈ dialkylaminosulfonyl, C₄-C₁₀ dialkylaminoalkyl, C₂-C₈ dialkylamino, C₃-C₁₀ trialkylsilyl, C₃-C₁₀ halotrialkylsilyl, C₂-C₇ cyanoalkyl, phenyl, pyridinyl, thienyl, naphthalenyl, phenoxy, phenylsulfonyl, pyridinylmethyl or benzyl; and

m is 0, 1, 2, 3, 4 or 5.

More particularly, this invention pertains to a compound of Formula 1 (including all geometric and stereoisomers), an N-oxide or a salt thereof.

This invention also relates to a fungicidal composition comprising a fungicidally effective amount of a compound of Formula 1 and at least one additional component selected from the group consisting of surfactants, solid diluents and liquid diluents.

This invention also relates to a fungicidal composition comprising a mixture of a compound of Formula 1 and at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises,” “comprising,” “includes,” “including,” “has,” “having”, “contains” or “containing” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl, hexyl or dodecyl isomers. “Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on alkoxy. Examples of “alkoxyalkoxy” include CH₃OCH₂O, CH₃OCH₂(CH₃)CHCH₂O and (CH₃)₂CHOCH₂CH₂O. Examples of “alkoxyalkoxyalkyl” include CH₃OCH₂OCH₂, CH₃CH₂O(CH₃)CHOCH₂ and (CH₃CH₂)₂OCH₂OCH₂.

“Alkylthio” includes branched or straight-chain alkylthio moieties such as methylthio, ethylthio, and the different propylthio, butylthio, pentylthio and hexylthio isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Alkylsulfinylalkyl” denotes alkylsulfinyl substitution on alkyl. Examples of “alkylsulfinylalkyl” include CH₃S(=O)CH₂, CH₃S(═O)CH₂CH₂, CH₃CH₂S(═O)CH₂ and CH₃CH₂S(═O)CH₂CH₂. “Alkylsulfonylalkyl” denotes alkylsulfonyl substitution on alkyl. Examples of “alkylsulfonylalkyl” include CH₃S(═O)₂CH₂, CH₃S(═O)₂CH₂CH₂, CH₃CH₂S(═O)₂CH₂ and CH₃CH₂S(═O)₂CH₂CH₂.

“Cyanoalkyl” denotes an alkyl group substituted with one cyano group. Examples of “cyanoalkyl” include NCCH₂, NCCH₂CH₂ and CH₃CH(CN)CH₂.

“Dialkylamino” includes an NH radical substituted with two alkyl groups each or which are independently straight-chain or branched and contain 1 to 4 carbon atoms. Examples of “dialkylamino”include (CH₃)₂N, (CH₃CH₂CH₂)₂N and CH₃CH₂(CH₃)N. Examples of “dialkylaminoalkyl” include ((CH₃)₂CH)₂NCH₂, (CH₃CH₂CH₂)₂NCH₂ and CH₃CH₂(CH₃)NCH₂CH₂.

“Dialkylamidino” denotes a straight-chain or branched dialkylamino bonded to the carbon atom of C(═NH)— moiety, or a straight-chain or branched alkylamino bonded to the carbon atom of a C(═N-alkyl)-moiety. Examples of “dialkylamidino” include (CH₃)₂NC(═NH)—, CH₃CH₂(CH₃)NC(═NH)— and CH₃NHC(═NCH₃)—. The term “dialkylimido” denotes two alkylcarbonyl groups each of which are independently straight-chain or branched and contain 2 to 6 carbon atoms bonded through a nitrogen atom. Examples of “dialkylimido” include (CH₃C(═O))₂N— and CH₃CH₂C(═O)(CH₃C(═O))N—. The term “pyridinylmethyl” denotes a pyridine ring bonded to the remainder of Formula 1 through a —CH₂— moiety. Said pyridine ring is attached to the —CH₂— moiety through any available carbon by replacement of a hydrogen on said carbon. The term “phenylsulfonyl” denotes a benzene ring bonded to the remainder of Formula 1 through a —SO₂— moiety. The term “thienyl” denotes a thiophene ring bonded to the remainder of Formula 1 through any available carbon by replacement of a hydrogen on said carbon.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl). The term “cycloalkoxy” denotes cycloalkyl linked through an oxygen atom such as cyclopentyloxy and cyclohexyloxy. The term “cycloalkoxyalkyl” denotes cycloalkoxy substitution on an alkyl moiety. Examples of “cycloalkoxyalkyl” include cyclopropoxymethyl, cyclopentoxyethyl, and other cycloalkoxy moieties bonded to straight-chain or branched alkyl groups. “Cycloalkylalkoxy” denotes cycloalkylalkyl linked through an oxygen atom. Examples of “cycloalkylalkoxy” include cyclopropylmethoxy, cyclopentylethoxy, and other cycloalkyl moieties bonded to straight-chain or branched alkoxy groups. “Alkylcycloalkylalkyl” denotes an alkyl group substituted with alkylcycloalkyl. Examples of “alkylcycloalkylalkyl” include 1-, 2-, 3- or 4-methyl or -ethyl cyclohexylmethyl. The term “cycloalkylthio” denotes cycloalkyl attached to and linked through a sulfur atom such as cyclopropylthio and cyclopentylthio; “cycloalkylsulfonyl” includes the corresponding sulfones.

“Alkoxycarbonyl” denotes a straight-chain or branched alkyloxy group bonded to a C(═O) moiety. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or octoxycarbonyl isomers. The term “alkoxycarbonylalkoxy” denotes alkoxycarbonyl substitution on straight-chain or branched alkoxy. Examples of “alkoxycarbonylalkoxy” include CH₃CH₂OC(═O)CH₂CH₂O and CH₃ CH₂CH(CH₃)OC(═O)CH₂O. “(Alkylcarbonyl)thio” denotes straight-chain or branched alkylcarbonyl attached to and linked through a sulfur atom. Examples of “(alkylcarbonyl)thio” include CH₃C(═O)S, CH₃CH₂CH₂C(═O)S and (CH₃)₂CHC(═O)S. “(Alkylthio)carbonyl” denotes a straight-chain or branched alkylthio group bonded to a C(═O) moiety. Examples of “(alkylthio)carbonyl” include CH₃SC(═O), CH₃CH₂CH₂SC(═O) and (CH₃)₂CHSC(═O). The term “cycloalkoxycarbonyl” means cycloalkoxy bonded to a C(═O) group, for example, cyclopropyloxycarbonyl and cyclopentyloxycarbonyl. “Cycloalkylalkoxycarbonyl” denotes a cycloalkylalkyl bonded to a OC(═O) moiety. Examples of “cycloalkylalkoxycarbonyl” include cyclopropyl-CH₂OC(═O), cyclopropyl-CH(CH₃)OC(═O) and cyclopentyl-CH₂OC(═O). Examples of “dialkylaminocarbonyl” include (CH₃)₂NC(═O), (CH₃CH₂)₂NC(═O), CH₃CH₂(CH₃)NC(═O), (CH₃)₂CHN(CH₃)C(═O) and CH₃CH₂CH₂(CH₃)NC(═O).

Trialkylsilyl” includes 3 branched and/or straight-chain alkyl radicals attached to and linked through a silicon atom, such as trimethylsilyl, triethylsilyl and tert-butyldimethylsilyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen”include F₃C, ClCH₂, CF₃CH₂ and CF₃CCl₂. The terms “haloalkoxy”, “haloalkylthio”, “haloalkylsulfinyl”, “haloalkylsulfonyl”, “halocycloalkyl”, “haloalkoxyalkoxy”, “haloalkoxyhaloalkoxy”, “halotrialkylsilyl and the like, are defined analogously to the term “haloalkyl”. Examples of “haloalkoxy” include CF₃O, CCl₃CH₂O, HCF₂CH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂. Examples of “halocycloalkyl” include 2-chlorocyclopropyl, 2-fluorocyclobutyl, 3-bromocyclopentyl and 4-chlorocyclohexyl. Examples of “haloalkoxyalkoxy” include CF₃OCH₂O, ClCH₂CH₂OCH₂CH₂O, Cl₃CCH₂OCH₂O as well as branched alkyl derivatives. Examples of “haloalkoxyhaloalkoxy” include CF₃OCHClO, ClCH₂CH₂OCHClCH₂O, Cl₃CCH₂OCHClO as well as branched alkyl derivatives. Examples of “halotrialkylsilyl” include CF₃(CH₃)₂Si, (CF₃)₃Si, and CH₂Cl(CH₃)₂Si.

The total number of carbon atoms in a substituent group is indicated by the “Cᵢ-Cⱼ” prefix where i and j are numbers from 1 to 14. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, said substituents (when they exceed 1) are independently selected from the group of defined substituents (e.g., (X)ₘ wherein m is 1, 2, 3, 4 or 5). When a variable group is shown to be optionally attached to a position, for example (X)ₘ wherein m may be 0, then hydrogen may be at the position even if not recited in the variable group definition. When one or more positions on a group are said to be “not substituted” or “unsubstituted”, then hydrogen atoms are attached to take up any free valency.

Unless otherwise indicated, a “ring” or “ring system” as a component of Formula 1 is carbocyclic or heterocyclic. The term “ring system” denotes two or more fused rings. The terms “bicyclic ring system” and “fused bicyclic ring system” denote a ring system consisting of two fused rings, in which either ring can be saturated, partially unsaturated, or fully unsaturated unless otherwise indicated. The term “heterobicyclic ring system” denotes a bicyclic ring system in which at least one ring atom is not carbon. The term “carbobyciclic ring system” denotes a bicyclic ring system in which all ring atoms are carbon. The term “ring member” refers to an atom or other moiety forming the backbone of a ring or ring system.

The terms “carbocyclic ring”, “carbocycle” or “carbocyclic ring system” denote a ring or ring system wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring”, “heterocycle” or “heterocyclic ring system” denote a ring or ring system in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen.

“Aromatic” indicates that each of the ring atoms is essentially in the same plane and has a p-orbital perpendicular to the ring plane, and that (4n+2) π electrons, where n is a positive integer, are associated with the ring to comply with Hückel's rule. The term “aromatic ring system” denotes a carbocyclic or heterocyclic ring system in which at least one ring of the ring system is aromatic. The term “aromatic carbocyclic ring system” denotes a carbocyclic ring system in which at least one ring of the ring system is aromatic. The term “aromatic heterocyclic ring system” denotes a heterocyclic ring system in which at least one ring of the ring system is aromatic. The term “nonaromatic ring system” denotes a carbocyclic or heterocyclic ring system that may be fully saturated, as well as partially or fully unsaturated, provided that none of the rings in the ring system are aromatic. The term “nonaromatic carbocyclic ring system” in which no ring in the ring system is aromatic. The term “nonaromatic heterocyclic ring system” denotes a heterocyclic ring system in which no ring in the ring system is aromatic.

The term “unsubstituted” in connection with a group such as a ring or ring system means the group does not have any substituents other than its one or more attachments to the remainder of Formula 1. The term “optionally substituted” in connection with a group such as a ring or ring system (e.g., 5- or 6-membered heteroaromatic ring of G¹ or G²) without specifying the number or identity of optional substituents refers to groups that are unsubstituted or have at least one non-hydrogen substituent that does not extinguish fungicidal activity of the unsubstituted analog. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 3.

The number of optional substituents may be restricted by an expressed limitation. For example, the phrase “optionally substituted with up to 4 substituents selected from R⁴ on carbon ring members means that 0, 1, 2, 3 or 4 substituents can be present (if the number of potential connection points allows). When a range specified for the number of substituents (e.g., r being an integer from 0 to 4 in Exhibit 1) exceeds the number of positions available for substituents on a ring (e.g., 3 positions available for (R^{V})ᵣ on U-2 in Exhibit 1), the actual higher end of the range is recognized to be the number of available positions.

When G¹ and G² is a nitrogen-containing heterocyclic ring or ring system, it may be attached to the remainder of Formula 1 through any available carbon or nitrogen ring atom, unless otherwise described.

As noted above, G¹ or G² can be (among others) phenyl optionally substituted with up to four substituents selected from a group of substituents as defined in the Summary of the Invention. An example of phenyl optionally substituted with up to four substituents is the ring illustrated as U-1 in Exhibit 1, wherein R^{v} is R⁴ as defined in the Summary of the Invention and r is an integer from 0 to 4.

As noted above, G¹ and G² can be (among others) a 5- to 6-membered heteroaromatic ring, optionally substituted with up to 4 substituents selected from a group of substituents as defined in the Summary of the Invention. Examples of an optionally substituted 5- to 6-membered heteroaromatic ring include the rings U-2 through U-61 illustrated in Exhibit 1 wherein R^{v} is any substituent as defined in the Summary of the Invention for G¹ and G² (i.e. R⁴ on carbon ring members and R⁵ on nitrogen ring members) and r is an integer from 0 to 4, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ. The optional substituents corresponding to R^{v} can be attached to any available carbon or nitrogen by replacing a hydrogen atom. The nitrogen atoms that require substitution to fill their valence are substituted with H or R^{v}.

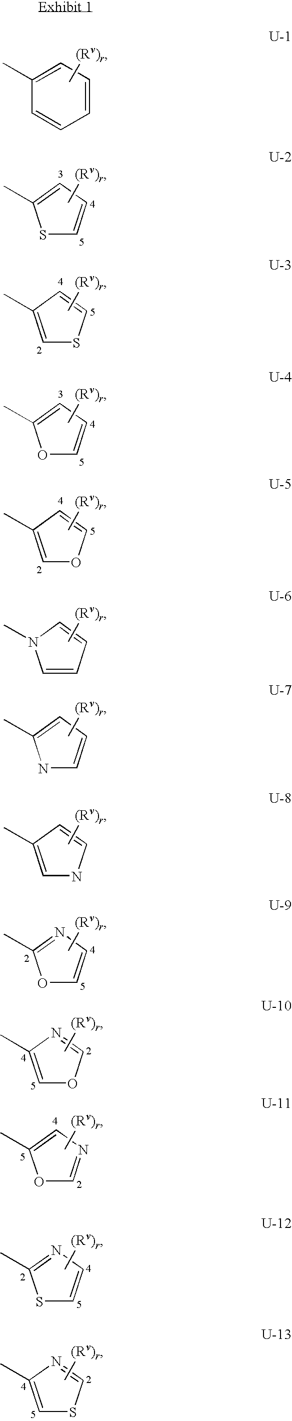

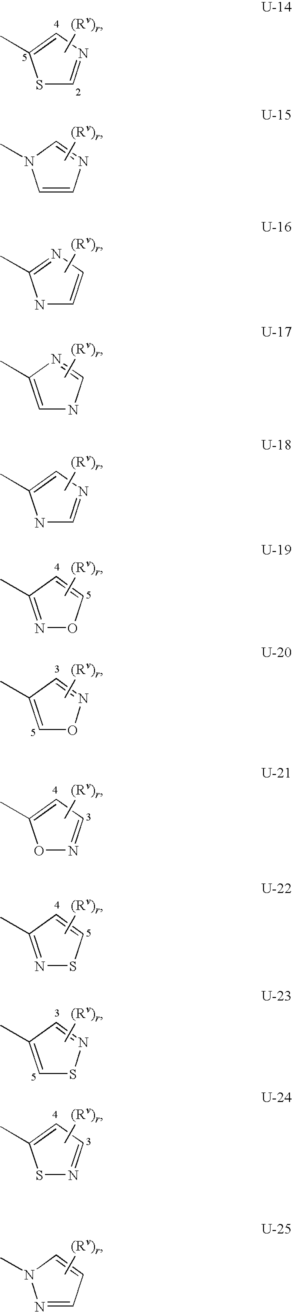

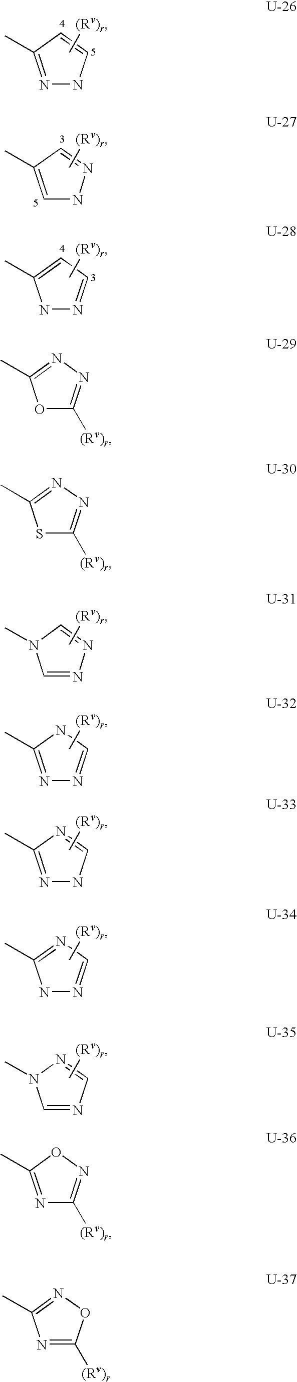

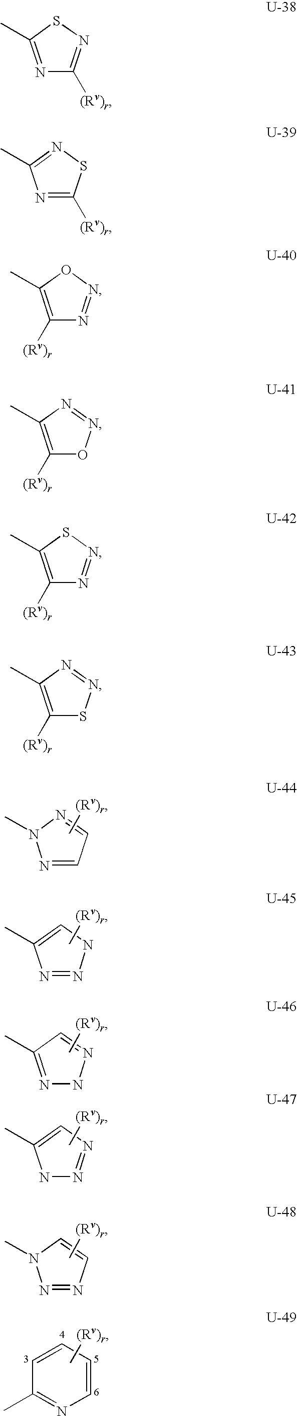

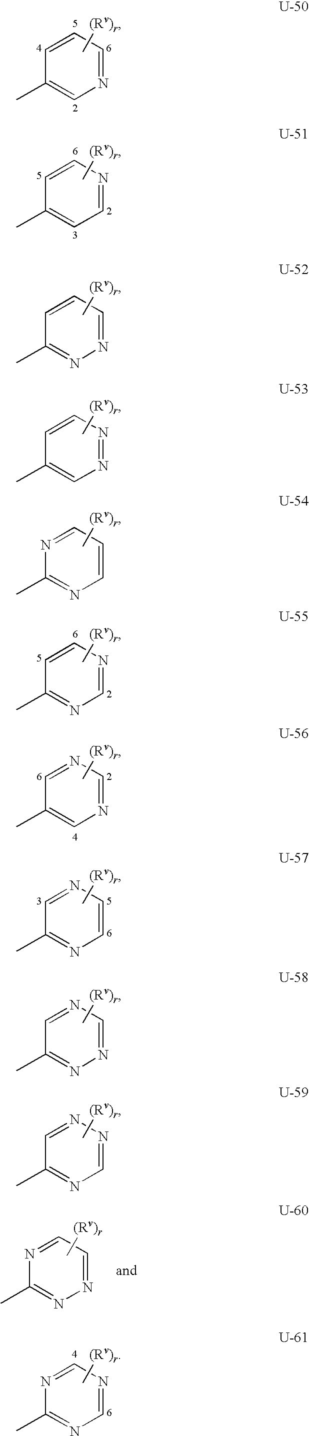

Also as noted above, G¹ and G² can be (among others) a 5- to 6-membered nonaromatic (i.e. saturated, partially unsaturated or fully unsaturated nonaromatic) heterocyclic ring optionally substituted with up to 4 substituents selected from the group of substituents as defined in the Summary of the Invention for G¹ and G² (i.e. R⁴ on carbon ring members and R⁵ on nitrogen ring members). Examples of a 5- to 6-membered nonaromatic heterocyclic ring include the rings K-1 through K-27 as illustrated in Exhibit 2. Note that when the attachment point on the K group is illustrated as floating, the K group can be attached to the remainder of Formula 1 through any available carbon or nitrogen of the K group by replacement of a hydrogen atom. The optional substituents corresponding to R^{v} (i.e. R⁴ on carbon ring members and R⁵ on nitrogen ring members) can be attached to any available carbon or nitrogen by replacing a hydrogen atom. For these K rings, r is an integer from 0 to 4, limited by the number of available positions on each G group. The nitrogen atoms that require substitution to fill their valence are substituted with H or R^{v}.

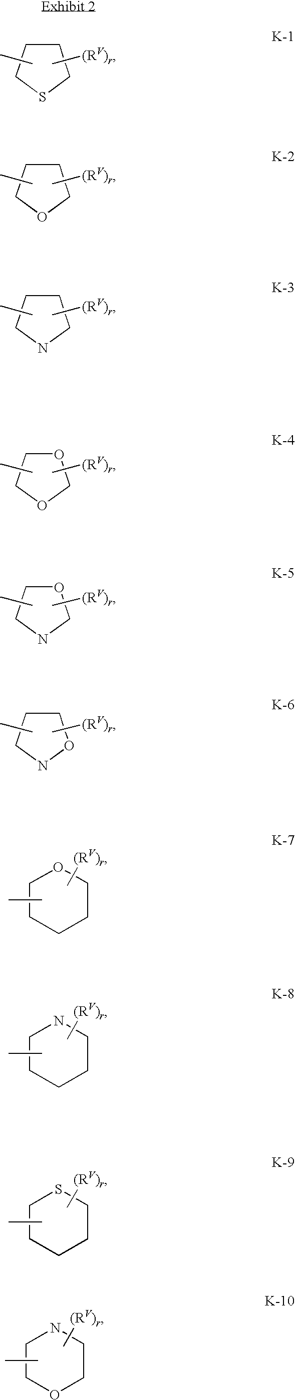

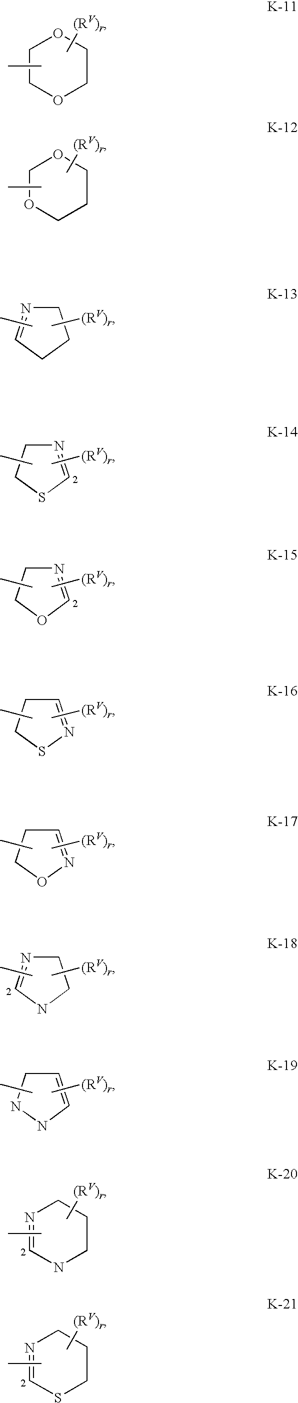

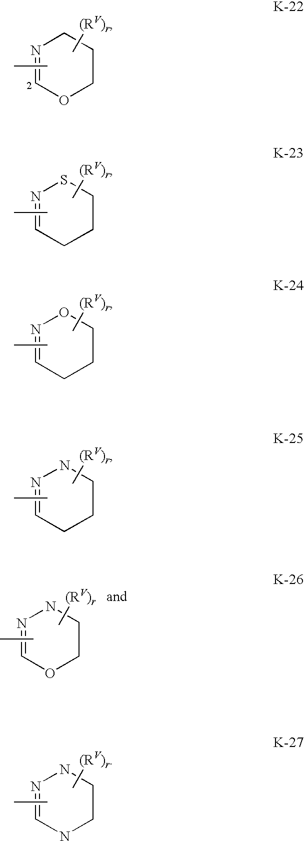

Further as noted above, G¹ and G² can be (among others) an 8- to 10-membered aromatic heterobicyclic ring system optionally substituted with up to 4 substituents selected from a group of substituents as defined in the Summary of the Invention. Examples of an 8- to 10-membered aromatic heterobicyclic ring system optionally substituted with up to 4 substituents include the rings J-1 through J-43 illustrated in Exhibit 3 wherein R^{v} is any substituent as defined in the Summary of the Invention for G¹ and G² (i.e. R⁴ on carbon ring members and R⁵ on nitrogen ring members), and r is an integer from 0 to 4. Note that when the attachment point on the J group is illustrated as floating, the J group can be attached to the remainder of Formula 1 through any available carbon or nitrogen of the J group by replacement of a hydrogen atom. The optional substituents corresponding to R^{v} can be attached to any available carbon or nitrogen by replacing a hydrogen atom. The nitrogen atoms that require substitution to fill their valence are substituted with H or R^{v}.

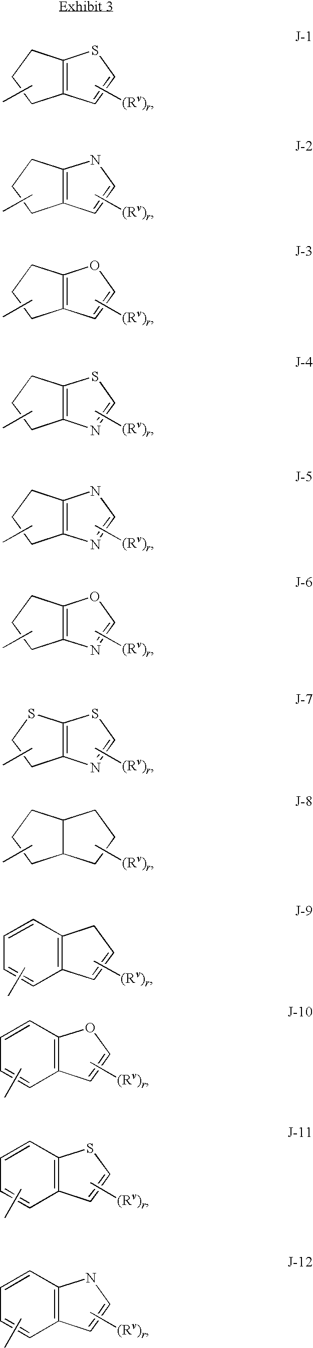

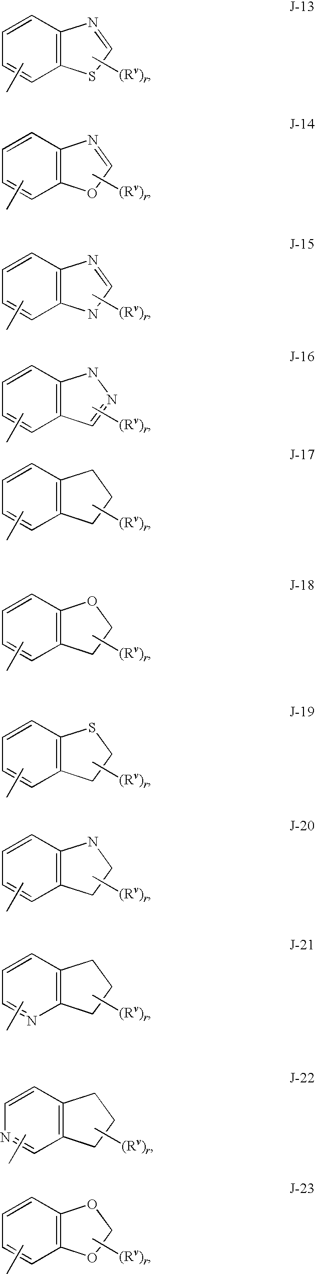

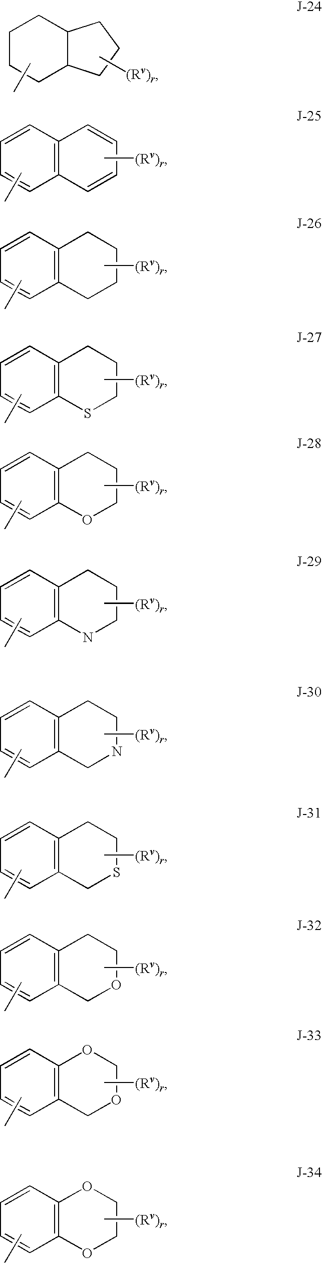

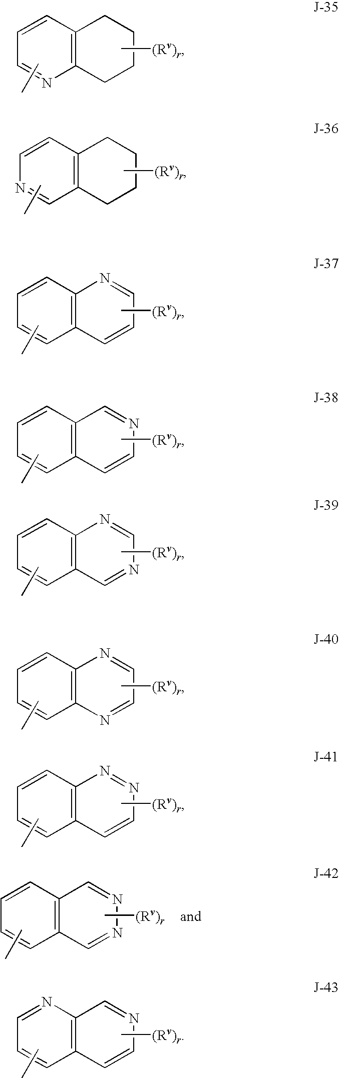

Structures U-18 through U-23, U-25 through U-34, and U-17 through U-42 also exemplify fused ring systems formed when a pair X substituents attached to contiguous carbon atoms are taken together with said carbon atoms to form a fused ring.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form. For example, Formula 1 possesses a chiral center at the 3-positon of the central isoxazolidine ring; and the two enantiomers at this position are depicted as Formula 1′ and Formula 1″ below wherein the chiral center is identified with an asterisk (*).

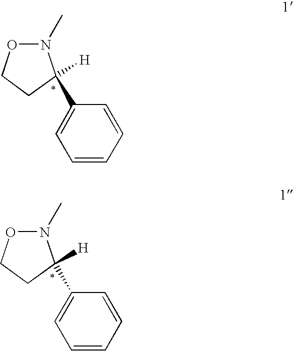

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

This invention comprises racemic mixtures, for example, equal amounts of the enantiomers of Formulae 1′ and 141 . In addition, this invention includes compounds that are enriched compared to the racemic mixture in an enantiomer of Formula 1. Also included are the essentially pure enantiomers of compounds of Formula 1, for example, Formula 1′ and Formula 1″.

When enantiomerically enriched, one enantiomer is present in greater amounts than the other, and the extent of enrichment can be defined by an expression of enantiomeric excess (“ee”), which is defined as (2x−1) 100%, where x is the mole fraction of the dominant enantiomer in the mixture (e.g., an ee of 20% corresponds to a 60:40 ratio of enantiomers).

Preferably the compositions of this invention have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of Formula 1 can comprise additional chiral centers. For example, substituents and other molecular constituents such as R¹, R², R³, X, R⁴ and R⁵ may themselves contain chiral centers. This invention comprises racemic mixtures as well as enriched and essentially pure stereoconfigurations at these additional chiral centers.

As noted above, compounds of Formula 1 may also be present as geometric isomers. A geometric isomer is defined as any of two or more stereoisomers that differ in the arrangement of atoms or groups of atoms around a structurally rigid bond, such as a double bond or, in the case of the present invention, a ring. Geometric isomers differ from one another in physical properties like melting and boiling points. For example the two aromatic groups in either Formula 2′ or Formula 2″ can be considered in a “cis” relationship to one another.

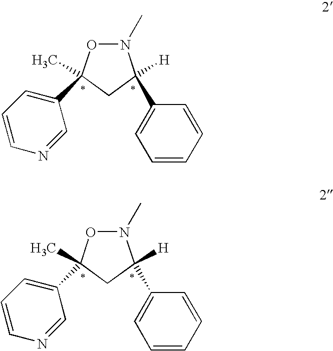

The geometric isomers of Formulae 2′ and 2″ are depicted below as Formulae 3′ and 3″ respectively. The two aromatic groups in either Formula 3′ or Formula 3″ can be considered in a “trans” relationship to one another.

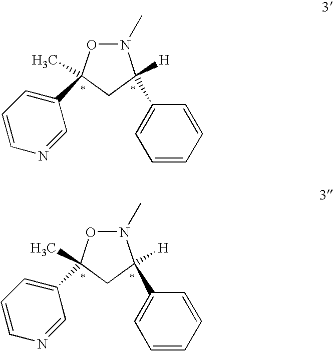

Separation of compounds of Formulae 2′ and 2″ (or Formulae 3′ and 3″) typically requires high resolution chromatographic methods. In contrast a compound of Formula 2′ from a compound of Formula 3′ (and similarly Formula 2″ from 3″) are more readily separated using chromatography. The geometric isomers of the compounds of Formula 1, are referred herein as “cis” and “trans” isomers with regards to the relative disposition of the aromatic rings attached to the isoxazoline ring. The “cis” geometric isomers of Formula 1 typically show enhanced fungicidal activity over their “trans” counterparts.

One skilled in the art will appreciate that not all nitrogen-containing heterocycles can form N-oxides since the nitrogen requires an available lone pair for oxidation to the oxide; one skilled in the art will recognize those nitrogen containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. Accordingly, the present invention comprises compounds selected from Formula 1 N-oxides and agriculturally suitable salts thereof.

Compounds selected from Formula 1, geometric and other stereoisomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.
Embodiment 1. A compound of Formula 1 wherein each A, B, and D is CH.
Embodiment 2. A compound of Formula 1 wherein A is N and B and D are both CH.
Embodiment 3. A compound of Formula 1 wherein B is N and A and D are both CH.
Embodiment 4. A compound of Formula 1 wherein D is N and A and B are both CH.
Embodiment 5. A compound of Formula 1 or any one of Embodiments 1 through 4 wherein each R¹ is independently H, C₁-C₃ alkyl, C₁-C₃ haloalkyl or C₃-C₅ cycloalkyl.
Embodiment 6. A compound of Embodiment 5 wherein R¹ is H, C₁-C₃ alkyl or C₁-C₃ haloalkyl.
Embodiment 7. A compound of Embodiment 6 wherein R¹ is H, CH₃ or CF₃.
Embodiment 8. A compound of Formula 1 or any one of Embodiments 1 through 7 wherein each R² is independently H, C₁-C₆ alkyl, C₃-C₅ cycloalkyl or C₄-C₆ cycloalkylalkyl.
Embodiment 8a. A compound of Embodiment 8 wherein R² is H or C₁-C₆ alkyl.
Embodiment 9. A compound of Embodiment 8a wherein R² is H or CH₃.
Embodiment 10. A compound of Embodiment 9 wherein R² is H.
Embodiment 11. A compound of Formula 1 or any one of Embodiments 1 through 10 wherein R³ is C₁-C₁₂ alkyl, —CH₂(C₁-C₆ haloalkyl), —CH₂(C₁-C₆ alkoxyalkyl), C₃-C₇ cycloalkyl, C₃-C₇ halocycloalkyl, C₃-C₇ cycloalkylalkyl or G¹.
Embodiment 12. A compound of Embodiment 11 wherein R³ is C₁-C₆ alkyl.
Embodiment 13. A compound of Embodiment 12 wherein R³ is CH₃.
Embodiment 14. A compound of Formula 1 or any one of Embodiments 1 through 13 wherein each instance of X is attached at a 3-, 4, or 5-position.
Embodiment 15. A compound of Formula 1 or Embodiment 14 wherein and an instance of X is attached at the 4-position.
Embodiment 16. A compound of Formula 1 or any of Embodiments 1 through 15 wherein each X that does not form a fused ring is independently halogen, cyano, nitro, C₁-C₆ alkyl, C₃-C₈ cycloalkyl, C₄-C₁₀ alkylcycloalkyl, C₄-C₁₀ cycloalkylalkyl, C₅-C₁₂ alkylcycloalkylalkyl, C₂-C₇ cyanoalkyl, C₂-C₈ alkoxyhaloalkyl, C₁-C₆ alkoxy, C₃-C₈ cycloalkoxy, C₄-C₁₀ cycloalkylalkoxy, C₁-C₆ alkylthio, C₃-C₈ cycloalkylthio, C₁-C₆ alkylsulfinyl, C₁-C₆ alkylsulfonyl, C₃₋C₈ cycloalkylsulfonyl, C₃-C₈ cycloalkylsulfinyl, C₂-C₈ alkoxycarbonyl, C₂-C₈ dialkylamino, C₄-C₁₂ dialkylimido, C₃-C₁₀ trialkylsilyl or G².
Embodiment 16a. A compound of Embodiment 16 wherein each X that does not form a fused ring is independently halogen, cyano, nitro, C₁-C₆ alkyl, C₃-C₈ cycloalkyl, C₄-C₁₀ alkylcycloalkyl, C₄-C₁₀ cycloalkylalkyl, C₅-C₁₂ alkylcycloalkylalkyl, C₂-C₇ cyanoalkyl, C₂-C₈ alkoxyhaloalkyl, C₁-C₆ alkoxy, C₃-C₈ cycloalkoxy, C₄-C₁₀ cycloalkylalkoxy, C₁-C₆ alkylthio, C₃-C₈ cycloalkylthio, C₁-C₆ alkylsulfinyl, C₁-C₆ alkylsulfonyl, C₃-C₈ cycloalkylsulfonyl, C₃-C₈ cycloalkylsulfinyl, C₂-C₈ dialkylamino, C₄-C₁₂ dialkylimido, C₃-C₁₀ trialkylsilyl or G².
Embodiment 17. A compound of Embodiment 16a wherein each X that does not form a fused ring is independently halogen, cyano, nitro, C₁-C₆ alkyl, C₃-C₈ cycloalkyl or G².
Embodiment 18. A compound of Embodiment 17 wherein each X that does not form a fused ring is independently halogen or G².
Embodiment 19. A compound of Formula 1 or any one of Embodiments 1 through 18 wherein no instance of X forms a fused ring.
Embodiment 20. A compound of Formula 1 or any one of Embodiments 1 through 18 wherein when a pair of X substituents attached to contiguous carbon atoms are taken together with said carbon atoms to form a fused ring, the fused ring is a fused phenyl or 5- to 6-membered nonaromatic carbocyclic ring optionally substituted with up to 4 substituents independently selected from R⁴ on carbon ring members.
Embodiment 21. A compound of Embodiment 20 wherein when a pair of X substituents attached to contiguous carbon atoms are taken together with said carbon atoms to form a fused ring, the fused ring is a fused phenyl ring optionally substituted with up to 4 substituents independently selected from R⁴ on carbon ring members.
Embodiment 22. A compound of Embodiment 21 wherein when a pair of X substituents attached to contiguous carbon atoms are taken together with said carbon atoms to form a fused ring, the fused ring is a fused phenyl ring.
Embodiment 23. A compound of Formula 1 or any one of Embodiments 1 through 22 wherein when a pair of X substituents attached to contiguous carbon atoms are taken together with said carbon atoms to form a fused ring, said X substituents are attached at the 3- and 4-positions or 4- and 5-positions.
Embodiment 24. A compound of Formula 1 or any one of Embodiments 1 through 23 wherein m is 0, 1, 2 or 3.
Embodiment 25. A compound of Embodiment 24 wherein m is 1, 2 or 3.
Embodiment 26. A compound of Embodiment 25 wherein m is 1 or 2.
Embodiment 27. A compound of Embodiment 26 wherein m is 1.
Embodiment 28. A compound of Formula 1 or any one of Embodiments 1 through 27 wherein each G¹ and G² is independently phenyl, benzyl, phenoxy, naphthalenyl or a 5- to 6-membered heteroaromatic ring, each optionally substituted with up to 4 substituents independently selected from R⁴ on carbon ring members and R⁵ on nitrogen ring members.
Embodiment 29. A compound of Embodiment 28 wherein each G¹ and G² is independently phenyl or a 5- to 6-membered heteroaromatic ring, each optionally substituted with up to 4 substituents independently selected from R⁴ on carbon ring members and R⁵ on nitrogen ring members.
Embodiment 30. A compound of Embodiment 29 wherein each G¹ and G² is phenyl or pyrazole, each optionally substituted with up to 4 substituents independently selected from R⁴ on carbon ring members and R⁵ on nitrogen ring members.
Embodiment 30a. A compound of Embodiment 30 wherein each G¹ and G² is pyrazole attached to Formula 1 through the 1-position of the pyrazole ring and optionally substituted with up to 4 substituents independently selected from R⁴ on carbon ring members and R⁵ on nitrogen ring members.
Embodiment 30b. A compound of Embodiment 30a wherein each G¹ and G² is pyrazole attached at the 4-positon of Formula 1 and through the 1-position of the pyrazole ring and optionally substituted with up to 4 substituents independently selected from R⁴ on carbon ring members and R⁵ on nitrogen ring members.
Embodiment 31. A compound of Formula 1 or any of Embodiments 1 through 30b where each R⁴ is independently halogen, cyano, nitro, C₁-C₆ alkyl, C₁-C₆ haloalkyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₂ alkylcycloalkylalkyl, C₂-C₈ alkoxyalkyl, C₂-C₈ haloalkoxyalkyl, C₂-C₈ alkoxyhaloalkyl, C₂-C₈ haloalkoxyhaloalkyl, C₄-C₁₀ cycloalkoxyalkyl, C₄-C₁₀ halocycloalkoxyalkyl, C₃₋C₁₀ alkoxyalkoxyalkyl, C₁-C₆ alkoxy, C₁-C₆ haloalkoxy, C₃-C8 cycloalkoxy, C₃-C₈ halocycloalkoxy, C₄-C₁₀ cycloalkylalkoxy, C₂-C₈ alkoxyalkoxy, C₂-C₈ haloalkoxyalkoxy, C₂-C₈ alkoxyhaloalkoxy, C₂-C₈ haloalkoxyhaloalkoxy, C₃-C₁₀ alkoxycarbonylalkoxy, C₂-C₈ alkylthioalkyl, C₁-C₆ alkylthio, C₁-C₆ haloalkylthio, C₂-C₈ alkoxycarbonyl, C₄-C₁₀ cycloalkoxycarbonyl, C₅-C₁₂ cycloalkylalkoxycarbonyl, C₂-C₈ alkylsulfonylalkyl, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₁₀ dialkylamidino, C₂-C₇ cyanoalkyl, phenyl, pyridinyl, thienyl, naphthalenyl, phenoxy or benzyl.
Embodiment 32. A compound of Embodiment 31 wherein each R⁴ is independently halogen, C₁-C₆ alkyl, C₁-C₆ haloalkyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₂ alkylcycloalkylalkyl, C₂-C₈ alkoxyalkyl, C₄-C₁₀ cycloalkoxyalkyl, C₁-C₆ alkoxy, C₁-C₆ haloalkoxy, C₃-C₈ cycloalkoxy, C₄-C₁₀ cycloalkylalkoxy, C₂-C₈ haloalkoxyalkoxy, C₂-C₈ alkoxyhaloalkoxy, C₃-C₁₀ alkoxycarbonylalkoxy, C₁-C₆ alkylthio, C₁-C₆ haloalkylthio, C₂-C₈ alkoxycarbonyl, C₄-C₁₀ cycloalkoxycarbonyl, C₅-C₁₂ cycloalkylalkoxycarbonyl, C₂-C₈ alkylsulfonylalkyl, C₁-C₆ haloalkylsulfonyl, phenyl, phenoxy or benzyl.
Embodiment 33. A compound of Embodiment 32 wherein each R⁴ is independently halogen, C₁-C₆ alkyl, C₁-C₆ haloalkyl, C₁-C₆ alkoxy, C₁-C₆ haloalkoxy, C₁-C₆ alkylthio, phenyl, phenoxy or benzyl.
Embodiment 34. A compound of Formula 1 or Embodiments 1 through 33 wherein each R⁵ is independently C₁-C₆ alkyl, C₁-C₆ haloalkyl, C₃-C₈ cycloalkyl, C₃-C₈ alkoxyalkyl, phenyl or pyridinyl.
Embodiment 35. A compound of Embodiment 34 wherein each R⁵ is independently C₁-C₆ alkyl or C₁-C₆ haloalkyl.
Embodiment 36. A compound of Embodiment 35 wherein R⁵ is CH₃.
Embodiment 37. A compound of Embodiment 1 wherein X is 4-(pyrazol-3-yl) and R⁵ is C₁-C₂ haloalkyl.

Embodiments of this invention, including Embodiments 1-37 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-37 above as well as any other embodiments described herein, and any combination thereof, pertain to the compositions and methods of the present invention.

Combinations of Embodiments 1-37 are illustrated by:
Embodiment A1. A compound of Formula 1 wherein
   R¹ is H, C₁-C₃ alkyl, C₁-C₃ haloalkyl or C₃-C₅ cycloalkyl;
   R² is H, C₁-C₆ alkyl, C₃-C₅ cycloalkyl or C₄-C₆ cycloalkylalkyl;
   R³ is C₁-C₁₂ alkyl, —CH₂(C₁-C₆ haloalkyl), —CH₂(C₁-C₆ alkoxyalkyl), C₃-C₇ cycloalkyl, C₃-C₇ halocycloalkyl, C₃-C₇ cycloalkylalkyl or G¹;
   each X is independently halogen, cyano, nitro, C₁-C₆ alkyl, C₃-C₈ cycloalkyl, C₄-C₁₀ alkylcycloalkyl, C₄-C₁₀ cycloalkylalkyl, C₅-C₁₂ alkylcycloalkylalkyl, C₂-C₇ cyanoalkyl, C₂-C₈ alkoxyhaloalkyl, C₁-C₆ alkoxy, C₃-C₈ cycloalkoxy, C₄-C₁₀ cycloalkylalkoxy, C₁-C₆ alkylthio, C₃-C₈ cycloalkylthio, C₁-C₆ alkylsulfinyl, C₁-C₆ alkylsulfonyl, C₃-C₈ cycloalkylsulfonyl, C₃-C₈ cycloalkylsulfinyl, C₂-C₈ dialkylamino, C₄-C₁₂ dialkylimido, C₃-C₁₀ trialkylsilyl or G²; or
   a pair of X substituents attached to contiguous carbon atoms are taken together with said carbon atoms to form a fused phenyl ring or 5- to 6-membered nonaromatic carbocyclic ring optionally substituted with up to 4 substituents independently selected from R⁴ on carbon ring members;
   each G¹ and G² is independently phenyl, benzyl, phenoxy, naphthalenyl or a 5- or 6-membered heteroaromatic ring, each optionally substituted with up to 4 substituents independently selected from R⁴ on carbon ring members and R⁵ on nitrogen ring members;
   each R⁴ is halogen, cyano, nitro, C₁-C₆ alkyl, C₁-C₆ haloalkyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₂ alkylcycloalkylalkyl, C₂-C₈ alkoxyalkyl, C₂-C₈ haloalkoxyalkyl, C₂-C₈ alkoxyhaloalkyl, C₂-C₈ haloalkoxyhaloalkyl, C₄-C₁₀ cycloalkoxyalkyl, C₄-C₁₀ halocycloalkoxyalkyl, C₃₋C₁₀ alkoxyalkoxyalkyl, C₁-C₆ alkoxy, C₁-C₆ haloalkoxy, C₃-C₈ cycloalkoxy, C₃-C₈ halocycloalkoxy, C₄-C₁₀ cycloalkylalkoxy, C₂-C₈ alkoxyalkoxy, C₂-C₈ haloalkoxyalkoxy, C₂-C₈ alkoxyhaloalkoxy, C₂-C₈ haloalkoxyhaloalkoxy, C₃-C₁₀ alkoxycarbonylalkoxy, C₂-C₈ alkylthioalkyl, C₁-C₆ alkylthio, C₁-C₆ haloalkylthio, C₂-C₈ alkoxycarbonyl, C₄-C₁₀ cycloalkoxycarbonyl, C₅-C₁₂ cycloalkylalkoxycarbonyl, C₂-C₈ alkylsulfonylalkyl, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₁₀ dialkylamidino, C₂-C₇ cyanoalkyl, phenyl, pyridinyl, thienyl, naphthalenyl, phenoxy or benzyl;
   each R⁵ is C₁-C₆ alkyl, C₁-C₆ haloalkyl, C₃-C₈ cycloalkyl, C₃-C₈ alkoxyalkyl, phenyl or pyridinyl; and
   m is 0, 1, 2, or 3.
Embodiment A2. A compound of Embodiment A1 wherein
   R¹ is H, C₁-C₃ alkyl or C₁-C₃ haloalkyl;
   R² is H or C₁-C₆ alkyl;
   R³ is C₁-C₆ alkyl;
   each X is independently halogen, cyano, nitro, C₁-C₆ alkyl, C₃-C₈ cycloalkyl or G²; or
   a pair of X substituents attached to contiguous carbon atoms are taken together with said carbon atoms to form a fused phenyl ring optionally substituted with up to 4 substituents independently selected from R⁴ on carbon ring members;
   each G² is independently phenyl or a 5- or 6-membered heteroaromatic ring, each optionally substituted with up to 4 substituents independently selected from R⁴ on carbon ring members and R⁵ on nitrogen ring members;
   R⁴ is halogen, C₁-C₆ alkyl, C₁-C₆ haloalkyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₂ alkylcycloalkylalkyl, C₂-C₈ alkoxyalkyl, C₄-C₁₀ cycloalkoxyalkyl, C₁-C₆ alkoxy, C₁-C₆ haloalkoxy, C₃-C₈ cycloalkoxy, C₄-C₁₀ cycloalkylalkoxy, C₂-C₈ haloalkoxyalkoxy, C₂-C₈ alkoxyhaloalkoxy, C₃₋C₁₀ alkoxycarbonylalkoxy, C₁-C₆ alkylthio, C₁-C₆ haloalkylthio, C₂-C₈ alkoxycarbonyl, C₄-C₁₀ cycloalkoxycarbonyl, C₅-C₁₂ cycloalkylalkoxycarbonyl, C₂-C₈ alkylsulfonylalkyl, C₁-C₆ haloalkylsulfonyl, phenyl, phenoxy or benzyl;
   each R⁵ is independently C₁-C₆ alkyl or C₁-C₆ haloalkyl; and
   m is 1, 2 or 3.
Embodiment A3. A compound in Embodiment A2 wherein
   A, B and D are CH,
   R¹ is H, CH₃ or CF₃;
   R² is H;
   R³ is CH₃;
   each X is halogen or G²; or
   a pair of X substituents attached to contiguous carbon atoms are taken together with said carbon atoms to form a fused phenyl ring, and said X substituents are attached at the 3- and 4-positions or 4- and 5-positions;
   each G² is phenyl or pyrazole, each optionally substituted with up to 4 substituents independently selected from R⁴ on carbon ring members and R⁵ on nitrogen ring members;
   each R⁴ is halogen, C₁-C₆ alkyl, C₁-C₆ haloalkyl, C₁-C₆ alkoxy, C₁-C₆ haloalkoxy, C₁-C₆ alkylthio, phenyl, phenoxy or benzyl;
   m is 1 or 2; and
   R⁵ is Me.
Embodiment A4. A compound in Embodiment A3 wherein
   each G² is pyrazole attached at the 4-positon of Formula 1 and through the 1-position of the pyrazole ring and optionally substituted with up to 4 substituents independently selected from R⁴ on carbon ring members and R⁵ on nitrogen ring members.
Embodiment A5. A compound in Embodiment A2 wherein
   A and B are CH, and D is N
   R¹ is H, CH₃ or CF₃;
   R² is H;
   R³ is CH₃;
   each X is halogen or G²; or
   two X attached to contiguous carbon atoms are taken together with said carbon atoms to form a fused phenyl ring, and said two X are attached at the 3- and 4-positions or 4- and 5-positions;
   each G² is phenyl or pyrazole, each optionally substituted with up to 4 substituents independently selected from R⁴ on carbon ring members and R⁵ on nitrogen ring members;
   each R⁴ is halogen, C₁-C₆ alkyl, C₁-C₆ haloalkyl, C₁-C₆ alkoxy, C₁-C₆ haloalkoxy, C₁-C₆ alkylthio, phenyl, phenoxy or benzyl;
   m is 1 or 2; and
   R⁵ is Me.

Specific Embodiments include compounds of Formula 1 selected from the group consisting of:
rel-3-[(3R,5S)-2,5-dimethyl-3-[4-[3-(trifluoromethyl)-1H-pyrazol-1-yl]phenyl]-5-isoxazolidinyl]pyridine,
rel-3-[(3R,5S)-3-[4-(4-bromo-1H-pyrazol-1-yl)phenyl]-2,5-dimethyl-5-isoxazolidinyl]pyridine,
rel-3-[(3R,5S)-3-(4-iodophenyl)-2-methyl-5-(trifluoromethyl)-5-isoxazolidinyl]pyridine,
rel-3-[(3R,5S)-2,5-dimethyl-3-(4′-methyl[1,1′-biphenyl]-4-yl)-5-isoxazolidinyl]pyridine;
rel-2-[(3R,5S)-3-[1,1′-biphenyl]-4-yl-2,5-dimethyl-5-isoxazolidinyl]pyrazine; and

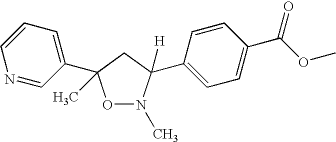

Of note are compounds of Formula 1 including geometric and stereoisomers, N-oxides, and salts thereof (including but not limited to Embodiments 1-37 and A1-A5 above) wherein each X is independently halogen, cyano, nitro, C₁-C₆ alkyl, C₃-C₈ cycloalkyl, C₄-C₁₀ alkylcycloalkyl, C₄-C₁₀ cycloalkylalkyl, C₅-C₁₂ alkylcycloalkylalkyl, C₂-C₇ cyanoalkyl, C₂-C₈ alkoxyhaloalkyl, C₁-C₆ alkoxy, C₃-C₈ cycloalkoxy, C₄-C₁₀ cycloalkylalkoxy, C₁-C₆ alkylthio, C₃-C₈ cycloalkylthio, C₁-C₆ alkylsulfinyl, C₁-C₆ alkylsulfonyl, C₃-C₈ cycloalkylsulfonyl, C₃-C₈ cycloalkylsulfinyl, C₂-C₈ dialkylaminosulfonyl.

Also of note are compounds of Formula 1 including geometric and stereoisomers, N-oxides, and salts thereof (including but not limited to Embodiments 1-37 and A1-A5 above) wherein a pair of X substituents attached to contiguous carbon atoms are taken together with said carbon atoms to form a fused phenyl ring, a fused 5- to 6-membered nonaromatic carbocyclic ring, a fused 5- or 6-membered heteroaromatic ring or a fused 5- to 6-membered nonaromatic heterocyclic ring, each fused ring optionally substituted with up to 4 substituents independently selected from R⁴.

This invention provides a fungicidal composition comprising a compound of Formula 1 (including all geometric and stereoisomers, N-oxides, and salts thereof), and at least one other fungicide. Of note as embodiments of such compositions are compositions comprising a compound corresponding to any of the compound embodiments described above.

This invention provides a fungicidal composition comprising a fungicidally effective amount of a compound of Formula 1 (including all geometric and stereoisomers, N-oxides, and salts thereof), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all geometric and stereoisomers, N-oxides, and salts thereof). Of note as embodiments of such methods are methods comprising applying a fungicidally effective amount of a compound corresponding to any of the compound embodiments described above. Of particular note are embodiments where the compounds are applied as compositions of this invention.

One or more of the following methods and variations as described in Schemes 1-5 can be used to prepare the compounds of Formula 1. In cases where a functional group is not compatible with the reaction conditions described for any of the reaction schemes, the group can be converted to a protected form prior to the described reaction and then deprotected after the reaction using commonly accepted protection/deprotection techniques (see Green, T. W and Wuts, P. G., Protecting Groups in Organic Transformations, 3rd Edition, John Wiley and Sons, New York, 1999). The definitions of A, B, D, R¹, R², R³, X and m in the compounds of Formulae 1-10 below are as defined above in the Summary of the Invention unless otherwise noted.

Compounds of Formula 1 can be prepared via a cycloaddition reaction of nitrones of Formula 2 and alkenes of Formula 3 as shown in Scheme 1. The reaction can be carried out in an inert solvent such as toluene or xylene at temperatures between about 20 to 130° C. for a period of time ranging from 1 to 120 h. The reaction can also be carried out at higher temperatures using a microwave reactor. In some cases addition of a catalyst can facilitate the reaction. A wide variety of catalyst can be useful in the present method including, for example, acetic acid, magnesium-based derivatives, cobalt(II) or (III) complexes, rhodium-based derivatives and hexafluoroantimonate. Typically these reactions result in mixtures of cis- and trans-isomers of Formula 1 which can be separated using chromatography. The method of Scheme 1 is illustrated in Step C of Examples 1, 2 and 3. Also the chemical literature describes related reactions, for references see Kanemasa et al., Chemistry Letters 1995, 1, 45-50; Rescifina et al., Journal of Medicinal Chemistry 2006, 49(2), 709-715 Tyrrell et al., Synthesis 2005, 14, 2393-2399; Fornefeld et al., Journal of Organic Chemistry 1979, 44(5), 835-839 and Dugovic et al., ARKIVOC 2003, XIV, 162-169.

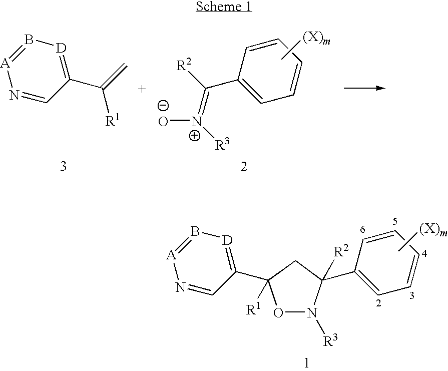

Nitrones of Formula 2 can be prepared by condensation of aldehydes of Formula 4 with hydroxylamine derivatives of Formula 5 (or its hydrochloride salt) as shown in Scheme 2. Preparation of nitrones is well documented in the chemical literature see, for example, Kumar et al., Synthetic Communications 2002, 32(12), 1887-1890; Tizot et al., Journal of Medicinal Chemistry 2000, 43(11), 2165-2175; Tyrrell et al., Synthesis 2005, 14, 2393-2399; Fornefeld et al., Journal of Organic Chemistry 1979, 44(5), 835-839; Knobloch et al., Organic Letters 2000, 2(8), 1117-1120; Chan et al., Journal of Organic Chemistry 1995, 60(6), 1741-1747; and Perrin et al. Journal of Organic Chemistry 1991, 56(25), 7034-7038. Also, the method of Scheme 2 is illustrated in Step B of Examples 1, 2 and 3.

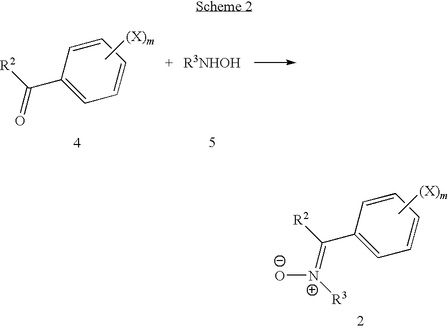

Hydroxylamine derivatives of Formula 5 are commercially available and can be prepared from the nitro precursors by methods well documented in the chemistry art.

Many alkenes of Formula 3 are commercially available and can be prepared by methods known to those skilled in the art. For example, using the method shown in Scheme 3, compounds of Formula 6 wherein X is bromo or iodo can be cross-coupled with compounds of Formula 7 (wherein M is H, Sn(Bu)₃, ZnCl, B(OH)₂ or other suitable counterion) in the presence of a palladium or nickel catalyst to produce compounds of Formula 3. Preferred catalysts include but are not limited to Pd(PPh₃)₄, PdCl₂(PPh₃)₂, PdCl₂(diphenylphosphinoferrocene), NiCl₂(PPh₃)₂ and tetrakis(tri-2-furylphosphino)-palladium. The exact conditions for each reaction will depend upon the catalyst used and the counterions in the compound of Formula 7. For relevant references see, for example, Fall et al., Synthesis 2007, 11, 1683-1696; Denmark et al., Journal of Organic Chemistry. 2006, 71(4), 1668-1676; Peyroux et al., Eur. J. Org. Chem. 2004, 5, 1075-1082; and Jiang et al., Tetrahedron Letters 2001, 42(24), 4083-4085.

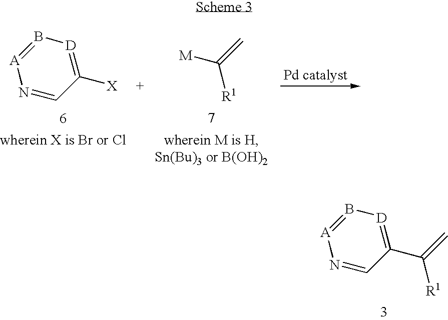

Alternatively, Compounds of Formula 3 can then be coupled with a boronic acid of Formula 8 using well-known Suzuki palladium-catalyzed cross coupling reaction conditions as shown in Scheme 4. Many catalysts are useful for the Suzuki reaction; particularly useful catalysts include tetrakis(triphenylphosphine)palladium(0), [1,1′-bis(diphenylphosphino)-ferrocene]dichloropalladium(II) and PdCl₂(PPh₃)₂. Solvents such as tetrahydrofuran, acetonitrile, diethyl ether and dioxane are suitable. Many boronic acids of Formula 8 are commercially available and others can be prepared by known methods. For a reference see, for example, Suzuki et al., Chemical Review 1995, 95, 2457-2483; WO2007/125984; Bethiol et al., Eur. J. Org. Chem. 2003, 6, 1091-1096; and Organ et al., J. Am. Chem. Soc. 2002, 124(7), 1288-1294.

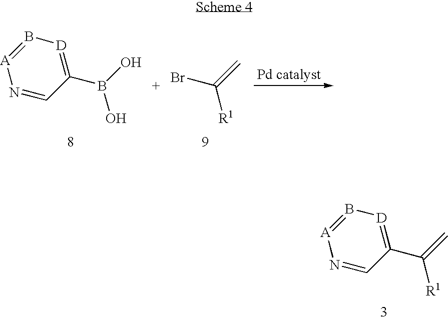

In another method, compounds of Formula 3 can be prepared by the reaction of a compound of Formula 10 with the compound of Formula 11 as shown in Scheme 5 using methods taught by Alunni et al., J. Org. Chem. 2003, 68(3), 718-725; Kobayashi et al., Heterocycles 2007, 71(8), 1827-1835; Kobayashi et al., Synthesis 2007, 6, 824-828; Takemiya et al., J. Am. Chem. Soc. 2006, 128(18), 6042-6043; Kim et al. Tetrahedron 2006, 62(17), 4120-4127; Imai et al., J. Org. Chem. 2004, 69(4), 1144-1150; and Tanaka et al., Org. Letters 2003, 5(8), 1365-1367.

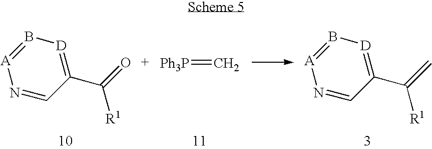

Many of compounds of Formulae 4, 6, 7, 9, 10 and 11 are commercially available and can be prepared by procedures generally known in the art.

It is recognized that some reagents and reaction conditions described may not be compatible with certain functionalities present in the intermediates. In these instances, the incorporation of protection/deprotection sequences into the synthesis will aid in obtaining the desired products. The use and choice of protecting groups will be apparent to one skilled in chemical synthesis.

One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “m” means multiplet and “br s” means broad singlet.

### EXAMPLE 1

### Preparation of rel-3-[(3R,5S)-3-(4-chlorophenyl)-2,5-dimethyl-5-isoxazolidinyl]pyridine (Compound 1) and rel-3-[(3R,5R)-3-(4-chlorophenyl)-2,5-dimethyl-5-isoxazolidinyl]pyridine (Compound 2)

### Step A: Preparation of 3-(1-methylethenyl)pyridine

A solution of potassium tert-butoxide in tetrahydrofuran (1.0 M, 125 mL, 125 mmol) was added dropwise to methyltriphenylphosphonium bromide (22.3 g, 62.5 mmol) at 0° C. under a nitrogen atmosphere with stirring, and then 3-acetylpyridine (5 mL, 50 mmol) was added. The reaction mixture was stirred at the room temperature for 12 h. Water (250 mL) was added to the reaction mixture, the organic layer was separated and the aqueous layer was extracted with ethyl acetate (200 mL). The organic layer was dried over MgSO₄, filtered and concentrated under reduced pressure. The resulting residue was purified by column chromatography on silica gel using 20-50% ethyl acetate in hexanes as eluant to give the title compound as an oil (3.8 g).

¹H NMR (CDCl₃) δ 2.17 (s, 3H), 5.18 (s, 1H), 5.42 (s, 1H), 7.24 (m, 1H), 7.72 (d, 1H), 8.50 (d, 1H), 8.72 (s, 1H).

### Step B: Preparation of [N(E/Z)]-N-[(4-chlorophenyl)methylene]methanamine N-oxide

To a solution of sodium hydroxide (0.48 g, 12 mmol) in water (20 mL) was added N-methylhydroxylamine hydrochloride (1.0 g, 12 mmol) and 4-chlorobenzaldehyde (1.6 g, 12 mmol). The reaction mixture was stirred at room temperature for 30 minutes, and then dichloromethane (50 mL) was added. The organic layer was separated and the aqueous layer was extracted with dichloromethane (2×50 mL). The combined organic layers were dried over MgSO₄, filtered and concentrated under reduced pressure to give the title compound as a white solid (2 g).

¹H NMR (CDCl₃) δ 3.87 (s, 3H), 7.35 (s, 1H), 7.55 (d, 2H), 8.11 (d, 2H).

### Step C: Preparation of rel-3-[(3R,5S)-3-(4-chlorophenyl)-2,5-dimethyl-5-isoxazolidinyl]pyridine (Compound 1) and rel-3-[(3R,5R)-3-(4-chlorophenyl)-2,5-dimethyl-5-isoxazolidinyl]pyridine (Compound 2)

To a mixture of [N(E/Z)]-N-[(4-chlorophenyl)methylene]methanamine N-oxide (i.e. the product of Step B) (2 g, 12 mmol) and acetic acid (0.4 g, 7 mmol) in toluene (12 mL) at about 70° C. under a nitrogen atmosphere was added a solution of 3-(1-methylethenyl)pyridine (i.e. the product of Step A) (1.3 g, 12 mmol) in toluene (10 mL). After the addition was complete, the reaction mixture was heated at reflux for 72 h, and then cooled to room temperature and concentrated under reduced pressure. The resulting residue was purified by column chromatography on silica gel using 10-50% ethyl acetate in hexanes as eluant to give rel-3-[(3R,5S)-3-(4-chlorophenyl)-2,5-dimethyl-5-isoxazolidinyl]pyridine, a compound of the present invention, as an oil (0.33 g), and rel-3-[(3R,5R)-3-(4-chlorophenyl)-2,5-dimethyl-5-isoxazolidinyl]pyridine, a compound of the present invention, as an orange oil (0.39 g).

Compound 1: ¹H NMR (CDCl₃) δ 1.63 (s, 3 H), 2.53-2.65 (m, 4 H), 2.82-2.91 (m, 1H), 3.70 (t, 1H), 7.09 (d, 2H), 7.21 (d, 2H), 7.30 (m, 1H), 7.90 (d, 1H), 8.53 (d, 1H), 8.71 (s, 1H).

Compound 2: ¹H NMR (CDCl₃) δ 1.77 (s, 3 H), 2.50-2.60 (m, 1 H), 2.67 (s, 3 H), 2.80-2.90 (m, 1H), 3.60 (t, 1H), 7.27-7.33 (m, 5 H), 7.81 (d, 1H), 8.53 (d, 1H), 8.78 (s, 1H).

### EXAMPLE 2

### Preparation of rel-3-[(3R,5S)-2,5-dimethyl-3-[4-[3-(trifluoromethyl)-1H-pyrazol-1-yl]phenyl]-5-isoxazolidinyl]pyridine (Compound 35) and rel-3-[(3R,5R)-2,5-dimethyl-3-[4-[3-(trifluoromethyl)-1H-pyrazol-1-yl]phenyl]-5-isoxazolidinyl]pyridine (Compound 36)

### Step A: Preparation of 4-[3-(trifluoromethyl)-1H-pyrazol-1-yl]benzaldehyde

A mixture of 3-(trifluoromethyl)pyrazole (13.2 g, 96.8 mmol), 4-fluorobenzaldehyde (8.5 mL, 81 mmol) and potassium carbonate (13.4 g, 96.8 mmol) in N,N-dimethylformamide (50 mL) was heated at 120° C. After for 12 h, water (200 mL) was added to the reaction mixture, and the aqueous layer was extracted with ethyl acetate (3×100 mL). The combined organic layers were washed with water (4×100 mL) and saturated aqueous sodium chloride solution (100 mL), dried over MgSO₄, filtered and concentrated under reduced pressure. The resulting residue was purified by column chromatography on silica gel using 1:4 ethyl acetate to hexanes as eluant to give the title compound as a white solid (6.4 g).

¹H NMR (CDCl₃) δ 6.79 (s, 1H), 7.93 (d, 2H), 8.03 (d, 2H), 8.06 (s, 1H), 10.06 (s, 1H).

### Step B: Preparation of [N(E/Z)]-N-[[4-[3-(trifluoromethyl)-1H-pyrazol-1-yl]phenyl]methylene]methanamine N-oxide

To a solution of sodium hydroxide (1.1 g, 28 mmol) in water (35 mL) was added N-methylhydroxylamine hydrochloride (2.3 g, 28 mmol) and 4-[3-(trifluoromethyl)-1H-pyrazol-1-yl]benzaldehyde (i.e. the product of Step A) (6.4 g, 27 mmol). The reaction mixture was stirred at room temperature for 12 h, and then water (100 mL) and ethyl acetate (100 mL) were added. The organic layer was separated, and the aqueous layer was extracted with ethyl acetate (100 mL). The organic layer washed with saturated aqueous sodium chloride solution (100 mL), dried over MgSO₄, filtered and concentrated under reduced pressure to give the title compound as an orange solid (5.6 g).

¹H NMR (CDCl₃) δ 3.92 (s, 3H), 6.75 (s, 1H), 7.43 (s, 1H), 7.79 (d, 2H), 8.01 (s, 1H), 8.37 (d, 2H).

### Step C: Preparation of rel-3-[(3R,5S)-2,5-dimethyl-3-[4-[3-(trifluoromethyl)-1H-pyrazol-1-yl]phenyl]-5-isoxazolidinyl]pyridine (Compound 35) and rel-3-[(3R,5R)-2,5-dimethyl-3-[4-[3-(trifluoromethyl)-1H-pyrazol-1-yl]phenyl]-5-isoxazolidinyl]pyridine (Compound 36)

To a mixture of [N(E/Z)]-N-[[4-[3-(trifluoromethyl)-1H-pyrazol-1-yl]phenyl]methylene]methanamine N-oxide (i.e. the product of Step B) (2.7 g, 10 mmol) and acetic acid (0.5 mL, 9 mmol) in toluene (12 mL) at about 70° C. under a nitrogen atmosphere was added a solution of 3-(1-methylethenyl)pyridine (i.e. the product of Step A of Example 1) (1.2 g, 10 mmol) in toluene (6 mL). The mixture was heated at reflux for 96 h, and then cooled to room temperature. Water (100 mL) and ethyl acetate (100 mL) were added to the reaction mixture, the organic layer was separated, and the aqueous layer was extracted with ethyl acetate (2×100 mL). The combined organic layers were dried over MgSO₄, filtered and concentrated under reduced pressure. The resulting residue was purified by column chromatography on silica gel using 20-70% ethyl acetate in hexanes as eluant to give rel-3-[(3R,5S)-2,5-dimethyl-3-[4-[3-(trifluoromethyl)-1H-pyrazol-1-yl]phenyl]-5-isoxazolidinyl]pyridine, a compound of the present invention, as an orange oil (88 mg) and rel-3-[(3R,5R)-2,5-dimethyl-3-[4-[3-(trifluoromethyl)-1H-pyrazol-1-5-isoxazolidinyl]pyridine, a compound of the present invention, as an oil (6 mg).

Compound 35: ¹H NMR (CDCl₃) δ 1.65 (s, 3H), 2.60-2.70 (m, 4H), 2.87-2.98 (m, 1H), 3.79 (t, 1H), 6.70 (s, 1H), 7.21-7.35 (m, 3H), 7.59 (d, 2H), 7.89 (br s, 2H), 8.53 (br s, 1H), 8.71 (s, 1H).

Compound 36: ¹H NMR (CDCl₃) δ 1.79 (s, 3H), 2.53-2.65 (m, 1H), 2.70 (s, 3H), 2.90 (m, 1H), 3.70 (t, 1H), 6.74 (s, 1H), 7.34 (br s, 1H), 7.51(d, 2H), 7.69 (d, 2H), 7.82 (d, 1H), 7.95 (s, 1H), 8.56 (br s, 1H), 8.79 (br s, 1H).

### EXAMPLE 3

### Preparation of rel-3-[(3R,5S)-3-(4-iodophenyl)-2-methyl-5-(trifluoromethyl)-5-isoxazolidinyl]pyridine (Compound 27) and rel-3-[(3R,5R)-3-(4-iodophenyl)-2-methyl-5-(trifluoromethyl)-5-isoxazolidinyl]pyridine (Compound 28)

### Step A: Preparation of 3-[1-(Trifluoromethyl)ethenyl]pyridine

A mixture of N,N-dimethylformamide (190 mL), water (90 mL) and potassium carbonate (37 g, 268 mmol) at room temperature was purged with nitrogen for about 10 minutes, and then added 2-bromo-3,3,3-trifluoro-1-propene (45 g, 256 mmol), 3-pyridineboronic acid (15 g, 122 mmol) and dichlorobis(triphenylphosphine)palladium(II) (3.4 g, 4.9 mmol) were added. The reaction flask was equipped with a dry ice condenser to prevent evaporation of the 2-bromo-3,3,3-trifluoro-1-propene and the reaction mixture was stirred at room temperature for 30 minutes, and then heat at about 60° C. for 2 h. The reaction mixture was allowed to cool to room temperature and stirred for 12 h. Water (200 mL) and petroleum ether (200 mL) were added to the reaction mixture, the mixture was filtered and the organic layer was separated. The aqueous layer was extracted with petroleum ether (2×200 mL). The combined organic layers were dried over MgSO₄, filtered and concentrated under reduced pressure. The resulting residue was purified by column chromatography on silica gel using dichloromethane as eluant to give the title compound as an orange oil (1.26 g).

¹H NMR (CDCl₃) δ 5.85 (s, 1H), 6.07 (s, 1H), 7.35 (m, 1H), 7.78 (d, 1H), 8.65 (d, 1H), 8.70 (s, 1H).

### Step B: Preparation of [N(E/Z)]-N-[(4-iodophenyl)methylene]methanamine N-oxide

To a solution of sodium hydroxide (0.96 g, 24 mmol) in water (30 mL) was added N-methylhydroxylamine hydrochloride (2.0 g, 24 mmol) and then 4-iodobenzaldehyde (5.3 g, 22.9 mmol). The reaction mixture was stirred at room temperature for 12 h, and then water (50 mL) and dichloromethane (50 mL) were added. The organic layer was separated and the aqueous layer was extracted with dichloromethane (2×50 mL). The combined organic layers were dried over MgSO₄, filtered and concentrated under reduced pressure to give the title compound as a white solid (6.0 g).

¹H NMR (CDCl₃) δ 3.87 (s, 3H), 7.32 (s, 1H), 7.75 (d, 2H), 7.95 (d, 2H).

### Step C: Preparation of Preparation of rel-3-[(3R,5S)-3-(4-iodophenyl)-2-methyl-5-(trifluoromethyl)-5-isoxazolidinyl]pyridine (Compound 27) and rel-3-[(3R,5R)-3-(4-iodophenyl)-2-methyl-5-(trifluoromethyl)-5-isoxazolidinyl]pyridine (Compound 28)

To a mixture of [N(E/Z)]-N-[(4-iodophenyl)methylene]methanamine N-oxide (i.e. the product of Step B) (0.52 g, 2.0 mmol) and acetic acid (0.30 mL, 5.2 mmol) in toluene (10 mL) at about 70° C. under a nitrogen atmosphere was added a solution of 3-[1-(trifluoromethyl)ethenyl]pyridine (i.e. the product of Step A) (0.34 g, 2.0 mmol) in toluene (3 mL). The reaction mixture was heated at reflux for 12 h, and then cooled to room temperature. The reaction mixture was concentrated under reduced pressure and the resulting residue was purified by column chromatography on silica gel using 10-25% ethyl acetate in hexanes as eluant to give rel-3-[(3R,5S)-3-(4-iodophenyl)-2-methyl-5-(trifluoromethyl)-5-isoxazolidinyl]pyridine, a compound of the present invention, as a solid (64 mg) melting at 80-83° C. and rel-3-[(3R,5R)-3-(4-iodophenyl)-2-methyl-5-(trifluoromethyl)-5-isoxazolidinyl]pyridine, a compound of the present invention, as a solid (150 mg) melting at 108-110° C.

Compound 27: ¹H NMR (CDCl₃) δ 2.65-2.71 (m, 4H), 3.35-3.40 (m, 1H), 3.80 (t, 1H), 7.00 (d, 2H), 7.37 (m, 1H), 7.64 (d, 2H), 7.92 (d, 1H), 8.65 (br s, 1H), 8.79 (br s, 1H).

Compound 28: ¹H NMR (CDCl₃) δ 2.71 (s, 3H), 2.92-3.00 (m, 1H), 3.07-3.15 (m, 1H), 3.51-3.58 (m, 1H), 7.11 (d, 2H), 7.37 (m, 1H), 7.70 (d, 2H), 7.90 (d, 1H), 8.65 (br s, 1H), 8.80 (s, 1H).

By the procedures described herein together with methods known in the art, the following compounds of Tables 1 to 9 can be prepared. The following abbreviations are used in the Tables which follow: t means tertiary, s means secondary, n means normal, i means iso, c means cyclo, Me means methyl, Et means ethyl, Pr means propyl, i-Pr means isopropyl, Bu means butyl, Ph means phenyl, OMe means methoxy, OEt means ethoxy, SMe means methylthio, SEt means ethylthio, Ph means phenyl and S(O)₂Me means methylsulfonyl.

TABLE 1

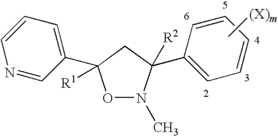

(X)ₘ
R¹ is H and R² is H.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-2-thienyl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is Me and R² is H.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-2-thienyl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is CF₃ and R² is H.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-2-thienyl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is H and R² is Me.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-2-thienyl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is Me and R² is Me.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-2-thienyl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is CF₃, and R² is Me.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-2-thienyl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)

TABLE 2

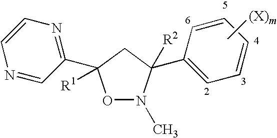

(X)ₘ
R¹ is Me and R² is H.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is CF₃ and R² is H
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is CF₃ and R² is Me.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)

TABLE 3

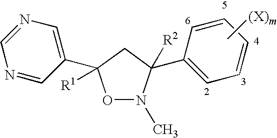

(X)ₘ
R¹ is Me and R² is H.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is CF₃ and R² is H.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R1 is CF₃ and R² is Me.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)

TABLE 4

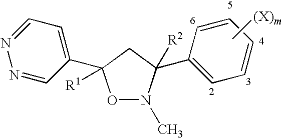

(X)ₘ
R¹ is Me and R² is H.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is CF₃ and R² is H.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)
R¹ is CF₃ and R² is Me.
4-CN
4-F
4-Cl
4-Br
4-I
4-Ph
4-Me
4-Et
4-i-Pr
4-n-Pr
4-n-Bu
4-i-Bu
4-c-pentyl
4-c-Pr
4-c-Bu
4-t-Bu
3,4-di-Cl
4-(4-fluorophenoxy)
4-(4-methylphenyl)
4-(4-ethylphenyl)
4-(4-i-propylphenyl)
4-(n-propylphenyl)
4-(3-CF₃-pyrazol-1-y)
4-(3-bromo-pyrazol-1-yl)
4-(4-Br)-pyrazol-1-yl
4-(3-Cl-pyrazol-1-yl)
4-(1,2,4-triazol-1-yl)
4-(3-I-1,2,4-triazol-1-yl)
4-(3-Et-1,2,4-triazol-1-yl)
4-(2-pyridinyl)
4-(4-pyrimidinyl)
4-(2-thienyl)
4-(4-Cl-imidazol-1-yl)
4-(4-CF₃-imidazol-1-yl)
4-NO₂
4-(4-Cl-pyrazol-1-yl)
4-(3-Me-pyrazol-1-yl)
4-(4-Me-pyrazol-1-yl)
4-(3,5-di-Me-pyrazol-1-yl)
4-(3,5-di-CF₃-pyrazol-1-yl)
4-(3-CF₃CF₂-pyrazol-1-yl)
4-(3-CF₃CF₂CF₂-pyrazol-1-yl)
4-(3-CF₃CF₂CF₂CF₂-pyrazol-1-yl)
4-(3-CF₃CF₂CF₂CF₂CF₂-pyrazol-1-yl)
4-(3-CF₃CF₂CF₂CF₂CF₂CF₂-pyrazol-1-yl)
4-(5-CF₃-thiophene-2-yl)
4-(5-Br-thiophene-2-yl)
4-c-hexyl
2-F
2-Br
4-phenoxy
4-(2,3-dimethylphenyl)
4-(3-phenyl-pyrazol-1-yl)
4-[3-(4-methylphenyl)-pyrazol-1-yl]
4-[3-(4-chlorophenyl)-pyrazol-1-yl]
4-[3-(4-bromophenyl)-pyrazol-1-yl]
4-(1-CF₃-pyrazol-3-yl)
4-(1-CHF₂-pyrazol-3-yl)
4-[1-(2,2,2-trifluoroethyl)-pyrazol-3-yl]
4-(5-Cl-2-thienyl)
4-(3-Cl-1,2,4-triazol-1-yl)
4-(3-CF₃-1,2,4-triazol-1-yl)
4-(3-phenyl-1,2,4-triazol-1-yl)
4-(3-pyridinyl)
4-(5-pyrimidinyl)
4-(imidazol-1-yl)
4-(4-Br-imidazol-1-yl)
4-(4-Me-imidazol-1-yl)
3-CN
4-(5-Me-2-thienyl)
4-(2-Cl-4-pyridinyl)
4-(2-Me-4-pyridinyl)
4-(2-Cl-5-pyrimidinyl)
4-(2-Me-5-pyrimidinyl)
4-OMe
4-OEt
4-SMe
4-SEt
4-N(Me)₂
4-S(O)₂Me
4-S(O)N(Me)₂
3-Br
3-I
2-Me
4-(4-OCF₃-phenyl)
4-(pyrazol-1-yl)
4-(2-Cl-3-pyridinyl)
4-(2-Me-3-pyridinyl)
4-(6-Cl-2-pyridinyl)
4-(6-Me-2-pyridinyl)
3-Cl
3-Me
3,4-dimethyl
3,5-dimethyl
4-(3-Br-1,2,4-triazol-1-yl)
4-(3-Me-1,2,4-triazol-1-yl)
4-(3,5-dichloro-1,2,4-triazol-1-yl)
4-(4-pyridinyl)
4-(2-pyrimidinyl)
4-(4-Ph-imidazol-1-yl)
4-(4-I-imidazol-1-yl)
4-(4-Et-imidazol-1-yl)

TABLE 5

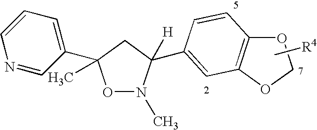

R⁴
7-Me
7-Br
7-c-Pr
7-c-Bu
5-Cl
7-Et
7-CF₃
7-OMe
7-t-Bu
5-Br
7-Cl
7-CHF₂
7-OEt
5-Me
5-CF₃

TABLE 6

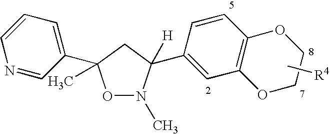

R⁴
8-Me
8-Br
8-c-Pr
7-Me
7-Br
7-c-Pr
5-Me
8-Et
8-CF₃
8-OMe
7-Et
7-CF₃
7-OMe
5-Cl
8-Cl
8-CHF₂
8-OEt
7-Cl
7-CHF₂
7-OEt
5-Br

TABLE 7

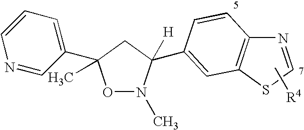

R⁴
7-Me
7-Br
7-c-Pr
5-Me
7-Et
7-CF₃
7-OMe
5-Cl
7-Cl
7-CHF₂
7-OEt
5-Br

TABLE 8

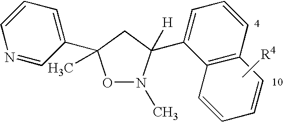

R⁴
4-Me
4-Br
4-c-Pr
10-Me
10-Br
10-c-Pr
4-Et
4-CF₃
4-OMe
10-Et
10-CF₃
10-OMe
4-Cl
4-CHF₂
4-OEt
10-Cl
10-CHF₂
10-OEt

TABLE 9

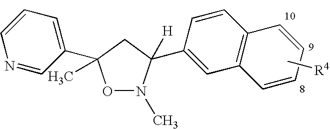

R⁴
9-Me
9-Br
9-c-Pr
8-Me
8-Br
8-c-Pr
10-Me
9-Et
9-CF₃
9-OMe
8-Et
8-CF₃
8-OMe
10-Cl
9-Cl
9-CHF₂
9-OEt
8-Cl
8-CHF₂
8-OEt
10-Br

### Formulation/Utility

A compound of this invention will generally be used as a fungicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

Weight Percent

| | Active | | |
|---|---|---|---|
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g, oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A-B. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### EXAMPLE A

High Strength Concentrate

| | |
|---|---|
| Compound 35 | 98.5% |
| silica aerogel | 0.5% |
| synthetic amorphous fine silica | 1.0% |

### EXAMPLE B

Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### EXAMPLE C

Granule

| | |
|---|---|
| Compound 13 | 10.0% |
| attapulgite granules (low volatile matter, | 90.0% |
| 0.71/0.30 mm; U.S.S. No. 25-50 sieves) | |

### EXAMPLE D

Extruded Pellet

| | |
|---|---|
| Compound 27 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### EXAMPLE E

Emulsifiable Concentrate

| | |
|---|---|
| Compound 35 | 10.0% |
| polyoxyethylene sorbitol hexoleate | 20.0% |
| C₆-C₁₀ fatty acid methyl ester | 70.0% |

### EXAMPLE F

Microemulsion

| | |
|---|---|
| Compound 27 | 5.0% |
| polyvinylpyrrolidone-vinyl acetate copolymer | 30.0% |
| Alkylpolyglycoside | 30.0% |
| glyceryl monooleate | 15.0% |
| Water | 20.0% |

### EXAMPLE G

Seed Treatment

| | |
|---|---|
| Compound 13 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| Polyorganosilane | 0.20% |
| colorant red dye | 0.05% |
| water | 65.75% |

Formulations such as those in the Formulation Table are typically diluted with water to form aqueous compositions before application. Aqueous compositions for direct applications to the plant or portion thereof (e.g., spray tank compositions) typically comprise at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondita, Puccinia striiformis, Puccinia hordei, Puccinia graminis and Puccinia arachidis), Hemileia vastatrix and Phakopsora pachyrhizi; other pathogens including Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these compounds can be influenced by many factors of the environment and should be determined under actual use conditions. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a fungicidally effective amount of a compound of Formula 1 and a biologically effective amount of at least one additional biologically active compound or agent and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phophorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, N-[2-(1S,2R)-[1,1′-bicyclopropyl]-2-ylphenyl]-3-(difluoromethyl)-1-methyl-1H-pyrazole-4-carboxamide and N-[2-(1,3-dimethylbutyl)phenyl]-5-fluoro-1,3-dimethyl-1H-pyrazole-4-carboxamide. The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (Qol) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071) and picoxystrobin. The methoxycarbamates include pyraclostrobin. The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen is an example of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16. 1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly. Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-S-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7) and (46.4) “benzophenone fungicides” (Fungicide Resistance Action Committee (FRAC) code U8). The thiazole carboxamides include ethaboxam. The phenyl-acetamides include cyflufenamid and N-[[(cyclopropylmethoxy)amino][6-(difluoromethoxy)-2,3-difluorophenyl]-methylene]benzeneacetamide. The quinazolinones include proquinazid and 2-butoxy-6-iodo-3-propyl-4H-1-benzopyran-4-one. The benzophenones include metrafenone. The (b46) class also includes bethoxazin, neo-asozin (ferric methanearsonate), pyrrolnitrin, quinomethionate, N-[2-[4-[[3-(4-chlorophenyl)-2-propyn-1-yl]oxy]-3-methoxy-phenyl]ethyl]-3-methyl-2-[(methylsulfonyl)amino]butanamide, N-[2-[4-[[3-(4-chloro-phenyl)-2-propyn-1-yl]oxy]-3-methoxyphenyl]ethyl]-3-methyl-2-[(ethylsulfonyl)amino]-butanamide, 2-[[2-fluoro-5-(trifluoromethyl)phenyl]thio]-2-[3-(2-methoxyphenyl)-2-thiazo-lidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl) [1,2,4]triazolo [1,5-a]pyrimidine, N-chloro-2-nitrophenyl)-N-ethyl-4-methylbenzenesulfonamide, N-[[(cyclopropylmethoxy)-amino][6-(difluoromethoxy)-2,3-difluorophenyl]methylene]benzeneacetamide, N′-[4-[4-chloro-3-(trifluoromethyl)phenoxy]-2,5-dimethylphenyl]-N-ethyl-N-methylmethanimid-amide and 1-[(2-propenylthio)carbonyl]-2-(1-methylethyl)-4-(2-methylphenyl)-5-amino-1H-pyrazol-3-one.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acequinocyl, acetamiprid, acrinathrin, amidoflumet, amitraz, avermectin, azadirachtin, azinphos-methyl, bifenthrin, bifenazate, bistrifluron, borate, 3-bromo-1-(3-chloro-2-pyridinyl)-N-[4-cyano-2-methyl-6-[(methylamino)carbonyl]phenyl]-1H-pyrazole-5-carboxamide, buprofezin, cadusafos, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenothiocarb, fenoxycarb, fenpropathrin, fenvalerate, fipronil, flonicamid, flubendiamide, flucythrinate, flufenerim, flufenoxuron, fluvalinate, tau-fluvalinate, fonophos, formetanate, fosthiazate, halofenozide, hexaflumuron, hexythiazox, hydramethylnon, imidacloprid, indoxacarb, insecticidal soaps, isofenphos, lufenuron, malathion, metaflumizone, metaldehyde, methamidophos, methidathion, methiodicarb, methomyl, methoprene, methoxychlor, metofluthrin, monocrotophos, methoxyfenozide, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spirodiclofen, spiromesifen, spirotetramat, sulprofos, tebufenozide, tebufenpyrad, teflubenzuron, tefluthrin, terbufos, tetrachlorvinphos, tetramethrin, thiacloprid, thiamethoxam, thiodicarb, thiosultap-sodium, tolfenpyrad, tralomethrin, triazamate, trichlorfon, triflumuron, Bacillus thuringiensis delta-endotoxins, entomopathogenic bacteria, entomopathogenic viruses and entomopathogenic fungi. The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

The following Tests demonstrate the control efficacy of compounds of this invention on specific pathogens. The pathogen control protection afforded by the compounds is not limited, however, to these species. See Index Tables A-B for compound descriptions. See Index Table C for ¹H NMR data. The following abbreviations are used in the Index Tables which follow: Me is methyl, Bu is butyl, Ph is phenyl, PhO is phenoxy and MeO is methoxy. The terms “cis” and “trans” refer to the geometric isomers assignment as previously described. The abbreviation “Ex.” stands for “Example” and is followed by a number indicating in which example the compound is prepared. The abbreviation “Geo.” stands for the geometric isomer (i.e. “cis” or “trans”).

INDEX TABLE A

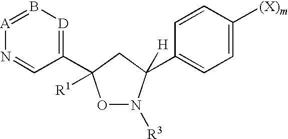

| Compd. | A | B | D | R¹ | R³ | Geo. | (X)ₘ | m.p. (° C.) |
|---|---|---|---|---|---|---|---|---|
| 1 | CH | CH | CH | CH₃ | CH₃ | cis | Cl | ** |
| (Ex. 1) | | | | | | | | |
| 2 | CH | CH | CH | CH₃ | CH₃ | trans | Cl | ** |
| (Ex. 1) | | | | | | | | |
| 3 | CH | CH | CH | CH₃ | CH₃ | cis | Br | * |
| 4 | CH | CH | CH | CH₃ | CH₃ | trans | Br | * |
| 7 | CH | CH | CH | CH₃ | CH₃ | cis | tert-Bu | * |
| 8 | CH | CH | CH | CH₃ | CH₃ | trans | tert-Bu | * |
| 9 | CH | CH | CH | CH₃ | CH₃ | cis | Ph-O | * |
| 10 | CH | CH | CH | CH₃ | CH₃ | trans | Ph-O | * |
| 11 | CH | CH | CH | CH₃ | CH₃ | cis | Ph | 109-111 |
| 12 | CH | CH | CH | CH₃ | CH₃ | trans | Ph | 101-102 |
| 13 | CH | CH | CH | CH₃ | CH₃ | cis | 4-Me-Ph | * |
| 14 | CH | CH | CH | CH₃ | CH₃ | trans | 4-Me-Ph | * |
| 15 | CH | CH | CH | CH₃ | CH₃ | cis | I | * |
| 16 | CH | CH | CH | CH₃ | CH₃ | trans | I | * |
| 17 | CH | CH | CH | CH₃ | CH₃ | cis | 4-(CF₃O)-Ph | * |
| 18 | CH | CH | CH | CH₃ | CH₃ | trans | 4-(CF₃O)-Ph | * |
| 19 | CH | CH | CH | CH₃ | CH₃ | cis | 4-F-Ph-O | * |
| 20 | CH | CH | CH | CH₃ | CH₃ | trans | 4-F-Ph-O | * |
| 21 | CH | CH | N | CH₃ | CH₃ | cis | Ph | * |
| 22 | CH | CH | N | CH₃ | CH₃ | trans | Ph | * |
| 23 | CH | CH | CH | CF₃ | CH₃ | cis | Ph | * |
| 24 | CH | CH | CH | CF₃ | CH₃ | trans | Ph | * |
| 25 | CH | CH | CH | CF₃ | CH₃ | cis | 4-Me-Ph | * |
| 26 | CH | CH | CH | CF₃ | CH₃ | trans | 4-Me-Ph | 112-114 |
| 27 | CH | CH | CH | CF₃ | CH₃ | cis | I | 80-83 |
| (Ex. 3) | | | | | | | | |
| 28 | CH | CH | CH | CF₃ | CH₃ | trans | I | 108-110 |
| (Ex. 3) | | | | | | | | |
| 29 | CH | CH | CH | CF₃ | CH₃ | cis | Br | 70-74 |
| 30 | CH | CH | CH | CF₃ | CH₃ | trans | Br | 130-131 |
| 31 | CH | CH | CH | CF₃ | CH₃ | cis | 2,3-di-Me-Ph | * |
| 32 | CH | CH | CH | CF₃ | CH₃ | trans | 2,3-di-Me-Ph | * |
| 33 | CH | CH | CH | CH₃ | CH₃ | cis | 1H-pyrazol-1-yl | * |
| 34 | CH | CH | CH | CH₃ | CH₃ | trans | 1H-pyrazol-1-yl | * |
| 35 | CH | CH | CH | CH₃ | CH₃ | cis | 3-CF₃-1H-pyrazol-1-yl | ** |
| (Ex. 2) | | | | | | | | |
| 36 | CH | CH | CH | CH₃ | CH₃ | trans | 3-CF₃-1H-pyrazol-1-yl | ** |
| (Ex. 2) | | | | | | | | |
| 37 | CH | CH | CH | CH₃ | CH₃ | cis | 4-Br-1H-pyrazol-1-yl | * |
| 38 | CH | CH | CH | CH₃ | CH₃ | trans | 4-Br-1H-pyrazol-1-yl | * |
| 39 | CH | CH | CH | CH₃ | CH₃ | cis | CH₃OC(═O) | * |
| 40 | CH | CH | CH | CH₃ | CH₃ | cis | 3-Ph-1H-pyrazol-1-yl | * |
| 41 | CH | CH | CH | CH₃ | CH₃ | trans | 3-Ph-1H-pyrazol-1-yl | * |
| 42 | CH | CH | CH | CH₃ | CH₃ | cis | 3-Me-1H-pyrazol-1-yl | * |
| 43 | CH | CH | CH | CH₃ | CH₃ | trans | 3-Me-1H-pyrazol-1-yl | * |
| 44 | CH | CH | CH | CF₃ | CH₃ | cis | 3-CF₃-1H-pyrazol-1-yl | * |
| 45 | CH | CH | CH | CF₃ | CH₃ | trans | 3-CF₃-1H-pyrazol-1-yl | * |
| 46 | CH | CH | CH | CH₃ | CH₃ | cis | 3-(2-thienyl)-1H-pyrazol-1-yl | * |
| 47 | CH | CH | CH | CH₃ | CH₃ | trans | 3-(2-thienyl)-1H-pyrazol-1-yl | * |
| 48 | CH | CH | CH | CH₃ | CH₃ | cis | 3-(4-Br-Ph)-1H-pyrazol-1-yl | 62-64 |
| 49 | CH | CH | CH | CH₃ | CH₃ | trans | 3-(4-Br-Ph)-1H-pyrazol-1-yl | 89-90 |
| 50 | CH | CH | CH | CH₃ | CH₃ | cis | 3,5-di-Me-1H-pyrazol-1-yl | * |
| 51 | CH | CH | CH | CH₃ | CH₃ | trans | 3,5-di-Me-1H-pyrazol-1-yl | * |
| 52 | CH | CH | CH | CH₃ | CH₃ | cis | 5-pyrimidinyl | * |
| 53 | CH | CH | CH | CH₃ | CH₃ | trans | 5-pyrimidinyl | * |
| 54 | CH | CH | CH | CH₃ | CH₃ | cis | 2-thienyl | 93-95 |
| 55 | CH | CH | CH | CH₃ | CH₃ | trans | 2-thienyl | * |
| 56 | CH | CH | CH | CH₃ | CH₃ | cis | 4-I-1H-pyrazol-1-yl | * |
| 57 | CH | CH | CH | CH₃ | CH₃ | trans | 4-I-1H-pyrazol-1-yl | * |
| 58 | CH | CH | CH | CH₃ | CH₃ | cis | 3,5-di-CF₃-1H-pyrazol-1-yl | * |
| 59 | CH | CH | CH | CH₃ | CH₃ | trans | 3,5-di-CF₃-1H-pyrazol-1-yl | 120-124 |
| 60 | CH | CH | CH | CH₃ | CH₃ | cis | 4-pyridinyl | * |
| 61 | CH | CH | CH | CH₃ | CH₃ | trans | 4-pyridinyl | * |
| 63 | CH | CH | CH | CH₃ | CH₃ | — | 1-(CF₃CH₂)-1H-pyrazol-5-yl | * |
| 69 | CH | CH | CH | CH₃ | CH₃ | cis | 4-Me-1H-pyrazol-1-yl | * |
| 70 | CH | CH | CH | CH₃ | CH₃ | cis | 5-(2-pyridinyl)-3-CF₃-1H-pyrazol-1-yl | * |
| 71 | CH | CH | CH | CH₃ | CH₃ | trans | 5-(2-pyridinyl)-3-CF₃-1H-pyrazol-1-yl | * |
| 72 | CH | CH | CH | H | CH₃ | cis | 3-CF₃-1H-pyrazol-1-yl | * |
| 73 | CH | CH | CH | H | CH₃ | trans | 3-CF₃-1H-pyrazol-1-yl | 82-85 |
| 74 | CH | CH | CH | CH₃ | CH₃ | cis | 3-(2-pyridinyl)-5-CF₃-1H-pyrazol-1-yl | * |
| 75 | CH | CH | CH | CH₃ | CH₃ | trans | 3-(2-pyridinyl)-5-CF₃-1H-pyrazol-1-yl | * |
| 80 | CH | CH | CH | CH₃ | CH₃ | — | 1-(CF₃CH₂)-1H-pyrazol-3-yl | * |

In the column “Geo.” cis and trans indicate isomeric configuration (as described above), and a dash (“—”) indicates a mixture of cis and trans isomers.
* See Index Table C for ¹H NMR data.
** See synthesis example for ¹H NMR data.

INDEX TABLE B

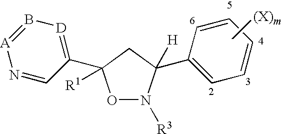

| Compd. | A | B | D | R¹ | R³ | Geo. | (X)ₘ | m.p. (° C.) |
|---|---|---|---|---|---|---|---|---|
| 5 | CH | CH | CH | CH₃ | CH₃ | trans | 3,4-di-Ci | * |
| 6 | CH | CH | CH | CH₃ | CH₃ | cis | 3,4-di-Ci | * |
| 62 | CH | CH | CH | CH₃ | CH₃ | — | 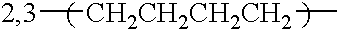 | * |
| 64 | CH | CH | CH | CH₃ | CH₃ | trans | 3-Br | |
| 65 | CH | CH | CH | CH₃ | CH₃ | cis | 2-Br | |
| 66 | CH | CH | CH | CH₃ | CH₃ | trans | 2-Br | |
| 67 | CH | CH | CH | CH₃ | CH₃ | cis | 3-Br | |
| 68 | CH | CH | CH | CH₃ | CH₃ | — | 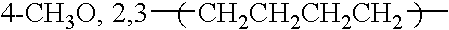 | * |
| 76 | CH | CH | CH | CH₃ | CH₃ | cis | 3-Cl | |
| 77 | CH | CH | CH | CH₃ | CH₃ | trans | 3-Cl | |
| 78 | CH | CH | CH | CH₃ | CH₃ | cis | 2-F | |
| 79 | CH | CH | CH | CH₃ | CH₃ | trans | 2-F | |
| 81 | CH | CH | CH | CH₃ | CH₃ | cis | 3,4-di-F | |
| 82 | CH | CH | CH | CH₃ | CH₃ | trans | 3,4-di-F | |
| 83 | CH | CH | CH | CH₃ | CH₃ | cis | 2,4-di-Br | |
| 84 | CH | CH | CH | CH₃ | CH₃ | trans | 2,4-di-Br | |
| 85 | CH | CH | CH | CH₃ | CH₃ | cis | 4-Br, 2-MeO | |
| 86 | CH | CH | CH | CH₃ | CH₃ | trans | 4-Br, 2-MeO | |
| 87 | CH | CH | CH | CH₃ | CH₃ | — | 3-I | |
| 88 | CH | CH | CH | CH₃ | CH₃ | — | 2-I | |
| 89 | CH | CH | CH | CH₃ | CH₃ | — | 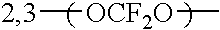 | |
| 90 | CH | CH | CH | CH₃ | CH₃ | — | 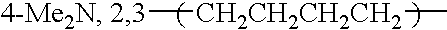 | |
| 91 | CH | CH | CH | CH₃ | CH₃ | cis | 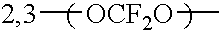 | |
| 92 | CH | CH | CH | CH₃ | CH₃ | cis | 2-Cl | |
| 93 | CH | CH | CH | CH₃ | CH₃ | trans | 2-Cl | |
| 94 | CH | CH | CH | CH₃ | CH₃ | cis | 3-F | |
| 95 | CH | CH | CH | CH₃ | CH₃ | trans | 3-F | |
| 96 | CH | CH | CH | CH₃ | CH₃ | trans | 4-Br, 2-F | |
| 97 | CH | CH | CH | CH₃ | CH₃ | cis | 4-Br, 2-F | |
| 98 | CH | CH | CH | CH₃ | CH₃ | — | 3-Me | |
| 99 | CH | CH | CH | CH₃ | CH₃ | — | 2-Me | |
| 100 | CH | CH | CH | CH₃ | CH₃ | — | 2-MeO | |
| 101 | CH | CH | CH | CH₃ | CH₃ | cis | 4-Br, 3-F | |

In the column “Geo.” cis and trans indicate isomeric configuration (as described above), and a dash (“—”) indicates a mixture of cis and trans isomers.
* See Index Table C for ¹H NMR data.
** See synthesis example for ¹H NMR data.

INDEX TABLE C

| Compd. | ¹H NMR Data (CDCl₃ solution unless indicated otherwise)^{a} |
|---|---|
| 3 | δ 1.63 (s, 3H), 2.53-2.65 (m, 4H), 2.82-2.91 (m, 1H), 3.70 (t, 1H), 7.03 (d, 2H), 7.30 (m, 1H), |
| | 7.38 (d, 2H), 7.87 (d, 1H), 8.53 (d, 1H), 8.71 (s, 1H). |
| 4 | δ 1.77 (s, 3H), 2.50-2.60 (m, 1H), 2.67 (s, 3H), 2.80-2.90 (m, 1H), 3.60 (t, 1H), 7.21-7.34 (m, 3H), |
| | 7.48 (d, 2H), 7.81 (d, 1H), 8.53 (d, 1H), 8.78 (s, 1H). |
| 5 | δ 1.68 (s, 3H), 2.35-2.40 (m, 1H), 2.73 (s, 3H), 3.08-3.15 (m, 1H), 4.19 (t, 1H), 7.21-7.35 (m, 2H), |
| | 7.40 (d, 1H), 7.69 (d, 1H), 7.82 (d, 1H), 8.53 (br s, 1H), 8.78 (br s, 1H). |
| 6 | δ 1.65 (s, 3H), 2.35-2.45 (m, 1H), 2.70 (s, 3H), 3.00-3.08 (m, 1H), 4.27 (t, 1H), 7.05 (t, 1H), |
| | 7.15 (d, 1H), 7.21-7.35 (m, 2H), 7.90 (d, 1H), 8.50 (br s, 1H), 8.71 (br s, 1H). |
| 7 | δ 1.27 (s, 9H), 1.64 (s, 3H), 2.60-2.71 (m, 4H), 2.81-2.89 (m, 1H), 3.70 (t, 1H), 7.08 (d, 2H), |
| | 7.21-7.35 (m, 3H), 7.90 (d, 1H), 8.50 (br s, 1H), 8.71 (s, 1H). |
| 8 | δ 1.32 (s, 9H), 1.79 (s, 3H), 2.56-2.65 (m, 1H), 2.68 (s, 3H), 2.78-2.87 (m, 1H), 3.60 (t, 1H), |
| | 7.27-7.30 (m, 5H), 7.81 (d, 1H), 8.53 (d, 1H), 8.78 (s, 1H). |
| 9 | δ 1.64 (s, 3H), 2.60-2.70 (m, 4H), 2.82-2.91 (m, 1H), 3.70 (t, 1H), 6.88 (d, 2H), 6.98 (d, 2H), |
| | 7.06-7.15 (m, 3H), 7.27-7.35 (m, 3H), 7.90 (d, 1H), 8.53 (d, 1H), 8.71 (s, 1H). |
| 10 | δ 1.79 (s, 3H), 2.56-2.65 (m, 1H), 2.69 (s, 3H), 2.80-2.90 (m, 1H), 3.60 (t, 1H), 6.95-7.06 (m, 4H), |
| | 7.08-7.15 (m, 1H), 7.27-7.40 (m, 5H), 7.81 (d, 1H), 8.54 (d, 1H), 8.78 (s, 1H). |
| 13 | δ 1.65 (s, 3H), 2.38 (s, 3H), 2.65-2.72 (m, 4H), 2.82-2.91 (m, 1H), 3.78 (t, 1H), 7.21-7.35 (m, 5H), |
| | 7.40-7.50 (m, 4H), 7.91 (d, 1H), 8.53 (br s, 1H), 8.72 (s, 1H). |
| 14 | δ 1.81 (s, 3H), 2.40 (s, 3H), 2.60-2.71 (m, 1H), 2.72 (s, 3H), 2.81-2.91 (m, 1H), 3.67 (t, 1H), |
| | 7.21-7.35 (m, 3H), 7.43 (d, 2H), 7.48 (d, 2H), 7.56 (d, 2H), 7.84 (d, 1H), 8.54 (d, 1H), 8.78 (s, 1H). |
| 15 | δ 1.63 (s, 3H), 2.53-2.65 (m, 4H), 2.82-2.91 (m, 1H), 3.67 (t, 1H), 6.90 (d, 2H), 7.30 (m, 1H), |
| | 7.58 (d, 2H), 7.87 (d, 1H), 8.53 (d, 1H), 8.71 (s, 1H). |
| 16 | δ 1.77 (s, 3H), 2.50-2.60 (m, 1H), 2.67 (s, 3H), 2.80-2.90 (m, 1H), 3.59 (t, 1H), 7.13 (d, 2H), |
| | 7.27-7.34 (m, 1H), 7.68 (d, 2H), 7.81 (d, 1H), 8.53 (d, 1H), 8.74 (s, 1H). |
| 17 | δ 1.66 (s, 3H), 2.65-2.72 (m, 4H), 2.88-2.92 (m, 1H), 3.78 (t, 1H), 7.21-7.35 (m, 5H), 7.43 (d, 2H), |
| | 7.54 (d, 2H), 7.92 (d, 1H), 8.53 (br s, 1H), 8.77 (br s, 1H). |
| 18 | δ 1.81 (s, 3H), 2.60-2.71 (m, 1H), 2.72 (s, 3H), 2.87-2.92 (m, 1H), 3.67 (t, 1H), 7.21-7.35 (m, 3H), |
| | 7.46 (d, 2H), 7.54 (d, 2H), 7.59 (d, 2H), 7.84 (d, 1H), 8.54 (br s, 1H), 8.79 (br s, 1H). |
| 19 | δ 1.64 (s, 3H), 2.60-2.70 (m, 4H), 2.82-2.91 (m, 1H), 3.70 (t, 1H), 6.85 (d, 2H), 6.90-7.08 (m, 4H), |
| | 7.10 (d, 2H), 7.27-7.35 (m, 1H), 7.90 (d, 1H), 8.51 (d, 1H), 8.71 (s, 1H). |
| 20 | δ 1.79 (s, 3H), 2.56-2.65 (m, 1H), 2.68 (s, 3H), 2.80-2.87 (m, 1H), 3.60 (t, 1H), 6.95-7.08 (m, 6H), |
| | 7.27-7.38 (m, 3H), 7.81 (d, 1H), 8.54 (d, 1H), 8.78 (s, 1H). |
| 21 | δ 1.68 (s, 3H), 2.67 (s, 3H), 2.81-2.87 (m, 1H), 2.91-3.00 (m, 1H), 3.78 (t, 1H), 7.18-7.60 (m, 9H), |
| | 8.49 (m, 2H), 9.12 (s, 1H). |
| 22 | δ 1.82 (s, 3H), 2.60-2.71 (m, 1H), 2.72 (s, 3H), 3.15-3.24 (m, 1H), 3.67 (t, 1H), 7.30-7.40 (m, 1H), |
| | 7.41-7.51 (m, 4H), 7.58 (m, 4H), 8.54 (m, 2H), 9.03 (s, 1H). |
| 23 | δ 2.71-2.81 (m, 4H), 3.37-3.46 (m, 1H), 3.90 (t, 1H), 7.27-7.58 (m, 10H), 7.98 (d, 1H), 8.65 (d, |
| | 1H), 8.81 (s, 1H). |
| 24 | δ 2.76 (s, 3H), 2.98-3.02 (m, 1H), 3.17-3.24 (t, 1H), 3.58-3.65 (m, 1H), 7.30-7.50 (m, 6H), 7.59 (m, |
| | 4H), 7.92 (d, 1H), 8.65 (d, 1H), 8.84 (s, 1H). |
| 25 | δ 2.39 (s, 3H), 2.71-2.81 (m, 4H), 3.37-3.46 (m, 1H), 3.90 (t, 1H), 7.21-7.56 (m, 9H), 7.98 (d, 1H), |
| | 8.65 (d, 1H), 8.81 (s, 1H). |
| 26 | δ 2.40 (s, 3H), 2.75 (s, 3H), 2.98-3.02 (m, 1H), 3.17-3.24 (t, 1H), 3.58-3.65 (m, 1H), 7.25-7.60 (m, |
| | 9H), 7.92 (d, 1H), 8.65 (d, 1H), 8.84 (s, 1H). |
| 29 | δ 2.65-2.71 (m, 4H), 3.35-3.40 (m, 1H), 3.81 (t, 1H), 7.12 (d, 2H), 7.37 (m, 1H), 7.44 (d, 2H), |
| | 7.92 (d, 1H), 8.65 (br s, 1H), 8.79 (br s, 1H). |
| 30 | δ 2.70 (s, 3H), 2.92-3.00 (m, 1H), 3.07-3.15 (t, 1H), 3.51-3.58 (m, 1H), 7.25 (d, 2H), 7.37 (m, 1H), |
| | 7.50 (d, 2H), 7.90 (d, 1H), 8.65 (d, 1H), 8.81 (s, 1H). |
| 31 | δ 2.29 (s, 3H), 2.31 (s, 3H), 2.71-2.81 (m, 4H), 3.37-3.46 (m, 1H), 3.90 (m, 1H), 7.18-7.40 (m, |
| | 6H), 7.51 (d, 2H), 7.98 (d, 1H), 8.65 (br s, 1H), 8.81 (br s, 1H). |
| 32 | δ 2.31 (s, 3H), 2.33 (s, 3H), 2.75 (s, 3H), 2.98-3.02 (m, 1H), 3.17-3.24 (t, 1H), 3.58-3.65 (m, 1H), |
| | 7.18-7.43 (m, 6H), 7.57 (d, 2H), 7.92 (d, 1H), 8.65 (br s, 1H), 8.84 (s, 1H). |
| 33 | δ 1.65 (s, 3H), 2.60-2.70 (m, 4H), 2.87-2.97 (m, 1H), 3.78 (t, 1H), 6.43 (s, 1H), 7.21-7.35 (m, 3H), |
| | 7.58 (d, 2H), 7.70 (s, 1H), 7.89 (m, 2H), 8.53 (br s, 1H), 8.71 (s, 1H). |
| 34 | δ 1.80 (s, 3H), 2.56-2.65 (m, 1H), 2.70 (s, 3H), 2.89 (m, 1H), 3.67 (t, 1H), 6.44 (s, 1H), 7.35 (br s, 1H), |
| | 7.48 (d, 2H), 7.65-7.72 (m, 3H), 7.82 (d, 1H), 7.95 (s, 1H), 8.56 (br s, 1H), 8.79 (br s, 1H). |
| 37 | δ 1.65 (s, 3H), 2.60-2.70 (m, 4H), 2.87-2.97 (m, 1H), 3.79 (t, 1H), 7.21-7.37 (m, 3H), 7.56 (d, |
| | 2H), 7.64 (s, 1H), 7.87 (m, 2H), 8.53 (d, 1H), 8.71 (s, 1H). |
| 38 | δ 1.79 (s, 3H), 2.56-2.65 (m, 1H), 2.70 (s, 3H), 2.89 (m, 1H), 3.67 (t, 1H), 7.27-7.35 (m, 1H), |
| | 7.49 (d, 2H), 7.60-7.72 (m, 3H), 7.82 (d, 1H), 7.95 (s, 1H), 8.56 (br s, 1H), 8.79 (br s, 1H). |
| 39 | δ 1.65 (s, 3H), 2.60 (m, 1H), 2.63 (s, 3H), 2.88 (m, 1H), 3.89 (s, 3H), 3.78 (m, 1H), 7.25 (m, 2H), |
| | 7.31 (m, 1H), 7.86 (m, 1H), 7.93 (m, 2H), 8.52 (m, 1H), 8.71 (m, 1H). |
| 40 | δ 1.66 (s, 3H), 2.60-2.70 (m, 4H), 2.87-2.97 (m, 1H), 3.79 (t, 1H), 6.75 (s, 1H), 7.21-7.45 (m, 6H), |
| | 7.65 (d, 2H), 7.9 (m, 4H), 8.53 (br s, 1H), 8.72 (s, 1H). |
| 41 | δ 1.81 (s, 3H), 2.56-2.65 (m, 1H), 2.71 (s, 3H), 2.89 (m, 1H), 3.67 (t, 1H), 6.79 (s, 1H), |
| | 7.27-7.53 (m, 6H), 7.75 (d, 2H), 7.95 (m, 4H), 8.55 (br s, 1H), 8.79 (s, 1H). |
| 42 | δ 1.65 (s, 3H), 2.35 (s, 3H), 2.60-2.70 (m, 4H), 2.82-2.92 (m, 1H), 3.75 (t, 1H), 6.20 (s, 1H), |
| | 7.21-7.35 (m, 3H), 7.53 (d, 2H), 7.78 (s, 1H), 7.9 (d, 1H), 8.53 (br s, 1H), 8.72 (s, 1H). |
| 43 | δ 1.80 (s, 3H), 2.38 (s, 3H), 2.56-2.65 (m, 1H), 2.69 (s, 3H), 2.87 (m, 1H), 3.65 (t, 1H), 6.24 (s, |
| | 1H), 7.30 (m, 1H), 7.43 (d, 2H), 7.63 (d, 2H), 7.78-7.92 (m, 2H), 8.53 (br s, 1H), 8.79 (s, 1H). |
| 44 | δ 2.72 (m, 4H), 3.40-3.45 (m, 1H), 3.91 (t, 1H), 6.71 (s, 1H), 7.35-7.40 (m, 3H), 7.65 (d, 2H), |
| | 7.95 (m, 2H), 8.63 (br s, 1H), 8.81 (s, 1H). |
| 45 | δ 2.73 (s, 3H), 3.02 (m, 1H), 3.15 (m, 1H), 3.61 (m, 1H), 6.75 (s, 1H), 7.38 (m, 1H), 7.53 (d, 2H), |
| | 7.72 (d, 2H), 7.95 (m, 2H), 8.63 (br s, 1H), 8.82 (s, 1H). |
| 46 | δ 1.65 (s, 3H), 2.60-2.70 (m, 4H), 2.87-2.97 (m, 1H), 3.79 (t, 1H), 6.63 (s, 1H), 7.05 (m, 1H), |
| | 7.21-7.35 (m, 4H), 7.40 (s, 1H), 7.61 (d, 2H), 7.9 (m, 2H), 8.53 (br s, 1H), 8.72 (s, 1H). |
| 47 | δ 1.80 (s, 3H), 2.56-2.67 (m, 1H), 2.70 (s, 3H), 2.87 (m, 1H), 3.67 (t, 1H), 6.70 (s, 1H), 7.08 (m, |
| | 1H), 7.21-7.35 (m, 2H), 7.41 (s, 1H), 7.48 (d, 2H), 7.71 (d, 2H), 7.85 (m, 1H), 7.92 (s, 1H), |
| | 8.53 (br s, 1H), 8.79 (s, 1H). |
| 50 | δ 1.65 (s, 3H), 2.27 (m, 6H), 2.60-2.70 (m, 4H), 2.82-2.92 (m, 1H), 3.78 (t, 1H), 5.98 (s, 1H), |
| | 7.21-7.45 (m, 5H), 7.90 (d, 1H), 8.53 (br s, 1H), 8.72 (s, 1H). |
| 51 | δ 1.80 (s, 3H), 2.29 (s, 3H), 2.31 (s, 3H), 2.56-2.65 (m, 1H), 2.7 (s, 3H), 2.87 (m, 1H), 3.65 (t, |
| | 1H), 6.0 (s, 1H), 7.21-7.45 (m, 5H), 7.85 (d, 1H), 8.53 (br s, 1H), 8.79 (s, 1H). |
| 52 | δ 1.66 (s, 3H), 2.60-2.70 (m, 4H), 2.87-2.97 (m, 1H), 3.80 (t, 1H), 7.27-7.35 (m, 3H), 7.48 (d, |
| | 2H), 7.9 (d, 1H), 8.53 (br s, 1H), 8.72 (s, 1H), 8.9 (s, 2H), 9.1 (s, 1H). |
| 53 | δ 1.81 (s, 3H), 2.6-2.7 (m, 1H), 2.73 (s, 3H), 2.87-2.97 (m, 1H), 3.73 (t, 1H), 7.27-7.35 (m, 1H), |
| | 7.57 (m, 4H), 7.85 (d, 1H), 8.53 (br s, 1H), 8.79 (s, 1H), 8.96 (s, 2H), 9.22 (s, 1H). |
| 55 | δ 1.79 (s, 3H), 2.56-2.67 (m, 1H), 2.69 (s, 3H), 2.87-2.92 (m, 1H), 3.67 (t, 1H), 7.27-7.35 (m, |
| | 2H), 7.48 (d, 2H), 7.63 (d, 2H), 7.71-7.96 (m, 3H), 8.53 (br s, 1H), 8.79 (s, 1H). |
| 56 | δ 1.65 (s, 3H), 2.60-2.70 (m, 4H), 2.87-2.97 (m, 1H), 3.79 (t, 1H), 7.21-7.37 (m, 3H), 7.53 (d, |
| | 2H), 7.69 (s, 1H), 7.87 (m, 2H), 8.53 (d, 1H), 8.71 (s, 1H). |
| 57 | δ 1.80 (s, 3H), 2.56-2.65 (m, 1H), 2.70 (s, 3H), 2.89 (m, 1H), 3.67 (t, 1H), 7.27-7.35 (m, 1H), |
| | 7.48 (d, 2H), 7.60-7.72 (m, 3H), 7.82 (d, 1H), 7.96 (s, 1H), 8.56 (br s, 1H), 8.79 (br s, 1H). |
| 58 | δ 1.66 (s, 3H), 2.60-2.70 (m, 4H), 2.91-2.98 (m, 1H), 3.80 (t, 1H), 7.05 (s, 1H), 7.27-7.40 (m, 5H), |
| | 7.87 (d, 1H), 8.53 (br s, 1H), 8.72 (s, 1H). |
| 60 | δ 1.66 (s, 3H), 2.60-2.70 (m, 4H), 2.87-2.97 (m, 1H), 3.79 (t, 1H), 7.27-7.35 (m, 3H), 7.4-7.57 (m, |
| | 4H), 7.9 (d, 1H), 8.5-8.8 (m, 4H). |
| 61 | δ 1.81 (s, 3H), 2.56-2.7 (m, 1H), 2.72 (s, 3H), 2.87-2.92 (m, 1H), 3.70 (t, 1H), 7.27-7.35 (m, 1H), |
| | 7.53 (m, 4H), 7.63 (d, 2H), 7.85 (d, 1H), 8.5-8.8 (m, 4H). |
| 62 | δ 8.8 and 8.72 (2 s, 1H), 8.55 and 8.44 (2 d, 1H), 8.09 and 7.98 (2 d, 1H), 7.70-7.90 (m, 3H), |
| | 7.42-7.52 (m, 3H), 7.22-7.36 (m, 2H), 4.40-4.56 (m, 1H), 3.02-3.15 (m, 1H), 2.80 and 2.73 (2 s, 3H), |
| | 2.61 (t, 1H), 1.78 and 1.71 (2 s, 3H). |
| 63 | δ 7.90 and 8.66 (2 s, 1H), 8.48-8.55 (m, 1H), 7.82-7.93 (m, 1H), 7.26-7.70 (m, 6H), 6.32 (d, 1H), |
| | 4.60-4.71 (m, 2H), 3.66-3.8 (m, 1H), 2.88-2.98 (m, 1H), 2.72 and 2.68 (2 s, 3H), 2.6-2.7 (m, 1H), |
| | 1.80 and 1.66 (2 s, 3H). |
| 68 | δ 8.8 and 8.72 (2 s, 1H), 8.5-8.58 (m, 1H), 8.26-8.34 (m, 1H), 7.96-8.06 (m, 1H), 7.84-7.90 (m, |
| | 1H), 7.4-7.52 (m, 2H), 7.26-7.34 (m, 2H), 6.81 and 6.68 (2 d, 1H), 4.32-4.44 (m, 1H), 4.0 and |
| | 3.94 (2 s, 3H), 2.95-3.08 (m, 1H), 2.77 and 2.68 (2 s, 3H), 2.55-2.64 (t, 1H), 1.78 and 1.70 (2 s, |
| | 3H). |
| 69 | δ 1.65 (s, 3H), 2.14 (s, 3H), 2.60-2.70 (m, 4H), 2.87-2.97 (m, 1H), 3.79 (t, 1H), 7.21-7.37 (m, 3H), |
| | 7.5-7.97 (m, 5H), 8.53 (br s, 1H), 8.72 (s, 1H). |
| 70 | δ 1.64 (s, 3H), 2.56-2.70 (m, 4H), 2.87-2.97 (m, 1H), 3.79 (t, 1H), 6.99 (s, 1H), 7.17-7.37 (m, 7H), |
| | 7.65 (m, 1H), 7.87 (d, 1H), 8.5-8.8 (m, 3H). |
| 71 | δ 1.77 (s, 3H), 2.53-2.65 (m, 1H), 2.68 (s, 3H), 2.81-2.97 (m, 1H), 3.67 (t, 1H), 7.02 (s, 1H), |
| | 7.17-7.71 (m, 8H), 7.82 (d, 1H), 8.5-8.6 (m, 2H), 8.79 (s, 1H). |
| 72 | δ 2.35-2.43 (m, 1H), 2.73 (s, 3H), 3.20-3.26 (m, 1H), 3.87 (t, 1H), 5.26-5.35 (m, 1H), 6.73 (s, 1H), |
| | 7.27-7.35 (m, 1H), 7.47 (d, 2H), 7.67 (d, 2H), 7.82 (d, 1H), 7.95 (s, 1H), 8.54 (br s, 1H), 8.65 (s, |
| | 1H). |
| 74 | δ 1.66 (s, 3H), 2.60-2.70 (m, 4H), 2.87-2.97 (m, 1H), 3.81 (t, 1H), 6.99 (s, 1H), 7.17-7.37 (m, 6H), |
| | 7.77 (m, 1H), 7.89 (d, 1H), 8.01 (d, 1H), 8.5-8.8 (m, 3H). |
| 75 | δ 1.80 (s, 3H), 2.56-2.70 (m, 1H), 2.73 (s, 3H), 2.81-2.97 (m, 1H), 3.75 (t, 1H), 7.27-7.60 (m, |
| | 7H), 7.77 (m, 1H), 7.85 (d, 1H), 8.01 (d, 1H), 8.5-8.8 (m, 3H). |
| 80 | δ 8.68-8.79 (m, 1H), 8.48-8.55 (m, 1H), 7.82-7.90 (m, 1H), 7.74-7.78 (m, 1H), 7.62-7.68 (m, 1H), |
| | 7.46-7.55 (m, 1H), 7.38-7.42 (m, 1H), 7.30-7.45 (m, 1H), 7.18-7.22 (m, 1H), 6.61 (d, 1H), |
| | 4.66-4.79 (m, 2H), 3.61-3.79 (m, 1H), 2.82-2.90 (m, 1H), 2.70 and 2.64 (2 s, 3H), 2.6-2.7 (m, 1H), |
| | 1.80 and 1.65 (2 s, 3H). |

^{a}¹H NMR data are in ppm downfield from tetramethylsilane. Couplings are designated by (s)-singlet, (d)-doublet, (t)-triplet, (m)-multiplet, (br s)-broad singlet.

### Biological Examples of the Invention

General protocol for preparing test suspensions for Tests A-I: The test compounds were first dissolved in acetone in an amount equal to 3% of the final volume and then suspended at the desired concentration (in ppm) in acetone and purified water (50/50 mix) containing 250 ppm of the surfactant Trem® 014 (polyhydric alcohol esters). The resulting test suspensions were then used in Tests A-I. Spraying a 200 ppm test suspension to the point of run-off on the test plants was the equivalent of a rate of 500 g/ha. (An asterisk “*” next to the rating value indicates a 40 ppm test suspension.)

### Test A

The test suspension was sprayed to the point of run-off on wheat seedlings. The following day the seedlings were inoculated with a spore dust of Erysiphe graminis f. sp. tritici (the causal agent of wheat powdery mildew) and incubated in a growth chamber at 20° C. for 8 days, after which time visual disease ratings were made.

### Test B

The test suspension was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 7 days, after which time visual disease ratings were made.

### Test C

The test suspension was sprayed to the point of run-off on wheat seedlings. The following day the seedlings were inoculated with a spore suspension of Septoria nodorum (the causal agent of wheat glume blotch) and incubated in a saturated atmosphere at 20° C. for 48 h, and then moved to a growth chamber at 20° C. for 7 days, after which time visual disease ratings were made.

### Test D

The test suspension was sprayed to the point of run-off on wheat seedlings. The following day the seedlings were inoculated with a spore suspension of Septoria tritici (the causal agent of wheat leaf blotch) and incubated in saturated atmosphere at 20° C. for 48 h, and moved to a growth chamber at 20° C. for 19 additional days, after which time visual disease ratings were made.

### Test E

The test suspension was sprayed to the point of run-off on tomato seedlings. The following day the seedlings were inoculated with a spore suspension of Botrytis cinerea (the causal agent of tomato Botrytis) and incubated in saturated atmosphere at 20° C. for 48 h, and then moved to a growth chamber at 24° C. for 3 additional days, after which time visual disease ratings were made.

### Test F

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 5 days, after which time visual disease ratings were made.

### Test G

The test suspension was sprayed to the point of run-off on creeping bent grass seedlings. The following day the seedlings were inoculated with a spore suspension of Rhizoctonia oryzae (the causal agent of turf brown patch) and incubated in a saturated atmosphere at 27° C. for 48 h, and then moved to a growth chamber at 27° C. for 3 days, after which time visual disease ratings were made.

### Test H

The test suspension was sprayed to the point of run-off on tomato seedlings. The following day the seedlings were inoculated with a spore suspension of Phytophthora infestans (the causal agent of tomato late blight) and incubated in a saturated atmosphere at 20° C. for 24 h, and then moved to a growth chamber at 20° C. for 4 days, after which time visual disease ratings were made

### Test I

Grape seedlings were inoculated with a spore suspension of Plasmopara viticola (the causal agent of grape downy mildew) and incubated in a saturated atmosphere at 20° C. for 24 h. After a short drying period, the test suspension was sprayed to the point of run-off on the grape seedlings and then moved to a growth chamber at 20° C. for 6 days, after which time the test units were placed back into a saturated atmosphere at 20° C. for 24 h. Upon removal, visual disease ratings were made.

Results for Tests A-I are given in Table A. In the table, a rating of 100 indicates 100 % disease control and a rating of 0 indicates no disease control (relative to the controls). A dash (−) indicates no test results. All results are for 200 ppm except where followed by “*” which indicates 40 ppm.

TABLE A

| Compd. | Test A | Test B | Test C | Test D | Test E | Test F | Test G | Test H | Test I |
|---|---|---|---|---|---|---|---|---|---|
| 1 | 97 | 0 | 100 | — | 83 | 0 | 0 | 0 | 0 |
| 2 | 72 | 0 | 99 | — | 0 | 0 | 0 | 0 | 17 |
| 3 | 93 | 0 | 100 | — | 79 | 0 | 0 | 0 | 0 |
| 4 | 90 | 32 | 100 | — | 80 | 0 | 0 | 0 | 0 |
| 5 | 98 | 0 | 0 | — | 91 | 51 | 0 | 0 | 0 |
| 6 | 99 | 0 | 0 | — | 0 | 0 | 0 | 0 | 17 |
| 7 | 99 | 34 | 69 | — | 99 | 40 | 0 | 0 | 0 |
| 8 | 99 | 69 | 40 | — | 56 | 0 | 0 | 0 | 0 |
| 9 | 99 | 82 | 95 | — | 99 | 78 | 0 | 17 | 0 |
| 10 | 93 | 82 | 98 | — | 98 | 0 | 0 | 0 | 0 |
| 11 | 99 | 91 | 97 | — | 99 | 26 | 0 | 0 | 0 |
| 12 | 99 | 74 | 82 | — | 29 | 0 | 0 | 0 | 0 |
| 13 | 99* | 0* | 0* | 92* | 99* | 0* | — | 0* | 0* |
| 14 | 91* | 0* | 0* | 0* | 0* | 0* | — | 0* | 0* |
| 15 | 99 | 0 | 99 | 98 | 98 | 100 | — | 0 | 0 |
| 16 | 99 | 0 | 95 | 96 | 56 | 0 | — | 0 | 0 |
| 17 | 100* | 0* | 0* | 30* | 94* | 15* | — | 0* | 0* |
| 18 | 0* | 0* | 0* | 58* | 0* | 7* | 0* | 0* | 0* |
| 19 | 98 | 91 | 99 | 98 | 98 | 7 | 0 | 0 | 0 |
| 20 | 91 | 92 | 78 | 92 | 0 | 0 | 0 | 0 | 0 |
| 21 | 97* | 0* | 0* | 95* | 0* | 62* | 0* | 0* | 0* |
| 22 | 86* | 0* | 0* | 97* | 0* | 0* | 0* | 0* | 0* |
| 23 | 99 | 0 | 97 | 98 | 0 | 4 | 0 | 0 | 0 |
| 24 | 99 | 45 | 0 | 100 | 0 | 0 | 0 | 0 | 0 |
| 25 | 100* | 0* | 0* | 86* | 71* | 0* | 0* | 0* | 0* |
| 26 | 96* | 0* | 0* | 75* | 0* | 0* | 0* | 0* | 0* |
| 27 | 100* | 0* | 100* | 95* | 0* | 100* | 0* | 0* | 0* |
| 28 | 99* | 0* | 33* | 88* | 0* | 0* | 0* | 0* | 0* |
| 29 | 98* | 0* | 67* | 76* | 0* | 83* | 0* | 0* | 0* |
| 30 | 100 | 89 | 92 | 83 | 0 | 0 | 0 | 0 | 58 |
| 31 | 98* | 0 | 0* | 50* | 0* | 0* | 0* | 0* | 0 |
| 32 | 97 | 0 | 0 | 85 | 0 | 0 | 0 | 0 | 9 |
| 33 | 99* | 0* | 99* | 80* | 39* | 58* | — | 0* | 0* |
| 34 | 98* | 0* | 99* | 88* | 56* | 6* | — | 0* | 0* |
| 35 | 100* | 100* | 100* | 99* | 99* | 98* | — | 0* | 0* |
| 36 | 79* | 0* | 0* | 73* | 22* | 23* | 7* | 0* | 0* |
| 37 | 95* | 74* | 99* | 99* | 94 | 93* | 0* | 0* | 0* |
| 38 | 0* | 0* | 0* | 77* | 0* | 42* | 0* | 0* | 0* |
| 39 | 88 | 0 | 51 | 32 | 90 | 0 | — | — | — |
| 40 | 78* | 0* | 64* | 77* | 75* | 0* | — | — | — |
| 41 | 78* | 0* | 0* | 65* | 0* | 0* | — | — | — |
| 42 | 71* | 9* | 100* | 88* | 99* | 97* | — | — | — |
| 43 | 0* | 0* | 90* | 79* | 88* | 52* | — | — | — |
| 44 | 100* | 19* | 69* | 89* | 0* | 0* | — | — | — |
| 45 | 96* | 0* | 0* | 52* | 0* | 0* | — | — | — |
| 46 | 0* | 0* | 0* | 62* | 78* | 0* | — | — | — |
| 47 | 35* | 0* | 0* | 42* | 0* | 0* | — | — | — |
| 48 | 61* | 0* | 0* | 43* | 20* | 0* | — | — | — |
| 49 | 61* | 0* | 0* | 27* | 0* | 0* | — | — | — |
| 50 | 0* | 0* | 87* | 73* | 84* | 0* | — | — | — |
| 51 | 0* | 0* | 0* | 32* | 0* | 0* | — | — | — |
| 52 | 0* | 0* | 99* | 76* | 92* | 59* | — | — | — |
| 53 | 0* | 0* | 0* | 68* | 25* | 31* | — | — | — |
| 54 | 71* | 9* | 98* | 79* | 99* | 99* | — | — | — |
| 55 | 61* | 0* | 84* | 93* | 97* | 85* | — | — | — |
| 56 | 52* | 0* | 99* | 74* | 94* | 91* | — | — | — |
| 57 | 0* | 0* | 0* | 57* | 0* | 0* | — | — | — |
| 58 | 80* | 0* | 0* | 91* | 0* | 8* | — | — | — |
| 59 | 52* | 0* | 0* | 2* | 0* | 0* | — | — | — |
| 60 | 0* | 0* | 0* | 3* | 29* | 0* | — | — | — |
| 61 | 0* | 0* | 0* | 5* | 0* | 0* | — | — | — |
| 62 | 97 | 17 | 97 | 80 | 88 | 91 | — | — | — |
| 63 | 21 | 0 | 0 | 36 | 0 | 8 | — | — | — |
| 64 | 64 | 0 | 0 | 45 | 0 | 0 | — | — | — |
| 65 | 0 | 0 | 0 | 5 | 0 | 0 | — | — | — |
| 66 | 0 | 0 | 0 | 5 | 0 | 0 | — | — | — |
| 67 | 47 | 0 | 0 | 8 | 34 | 0 | — | — | — |
| 68 | 0 | 0 | 0 | 65 | 0 | 0 | — | — | — |
| 69 | 0* | 0* | 97* | 12* | 87* | 99* | — | — | — |
| 70 | 0* | 26* | 0* | 54* | 0* | 72* | — | — | — |
| 71 | 0* | 28* | 0* | 10* | 0* | 12* | — | — | — |
| 72 | 99* | 100* | 100* | 93* | 96* | 94* | — | — | — |
| 73 | 94* | 100* | 99* | 71* | 95* | 82* | — | — | — |
| 74 | 0* | 22* | 0* | 2* | 0* | 0* | — | — | — |
| 75 | 0* | 22* | 0* | 19* | 0* | 0* | — | — | — |
| 76 | 84 | 0 | 0 | 34 | 85 | 9 | — | — | — |
| 77 | 0 | 0 | 0 | 62 | 0 | 0 | — | — | — |
| 78 | 0 | 0 | 0 | 16 | 0 | 0 | — | — | — |
| 79 | 0 | 27 | 22 | 7 | 0 | 0 | — | — | — |
| 80 | 0 | 0 | 0 | 94 | 0 | 0 | — | — | — |
| 81 | 56 | 0 | 0 | 15 | 78 | 0 | — | — | — |
| 82 | 0 | 18 | 0 | 40 | 0 | 0 | — | — | — |
| 83 | 98 | 0 | 81 | 91 | 82 | 56 | — | — | — |
| 84 | 94 | 27 | 60 | 52 | 43 | 0 | — | — | — |
| 85 | 90 | 0 | 64 | 89 | 0 | 92 | — | — | — |
| 86 | 91 | 9 | 0 | 76 | 0 | 0 | — | — | — |
| 87 | 0 | 0 | 0 | 84 | 0 | 0 | — | — | — |
| 88 | 81 | 27 | 0 | 70 | 0 | 0 | — | — | — |
| 89 | 0 | 0 | 0 | 36 | 0 | 16 | — | — | — |
| 90 | 0 | 0 | 0 | 35 | 0 | 0 | — | — | — |
| 91 | 0 | 0 | 0 | 0 | 0 | 0 | — | — | — |
| 92 | 0 | 0 | 0 | 22 | 0 | 0 | — | — | — |
| 93 | 0 | 0 | 0 | 3 | 0 | 0 | — | — | — |
| 94 | 84 | 0 | 0 | 4 | 0 | 0 | — | — | — |
| 95 | — | — | — | — | — | — | — | — | — |
| 96 | 48 | 0 | 78 | 77 | 0 | 0 | — | — | — |
| 97 | — | — | — | — | — | — | — | — | — |
| 98 | 0* | 0* | 0* | 3* | 0* | 0* | — | — | — |
| 99 | 0 | 17 | 0 | 18 | 0 | 0 | — | — | — |
| 100 | 21 | 0 | 0 | 13 | 26 | 8 | — | — | — |
| 101 | 99 | 0 | 86 | 68 | 100 | 99 | — | — | — |
| 102 | 92 | 17 | 0 | 85 | 62 | 8 | — | — | — |
| 103 | 0 | 17 | 0 | 32 | 32 | 31 | — | — | — |
| 104 | 63* | 0* | 0* | 82* | 0* | 0* | — | — | — |
| 105 | 0* | 0* | 0* | 62* | 0* | 0* | — | — | — |

What is claimed is:

1. A compound selected from Formula 1, N-oxides and salts thereof, 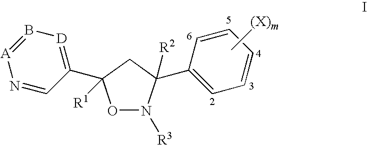
wherein
each A, B and D is independently N or CH,
provided that no more than one of A, B and D is N;
R¹ is H, C₁-C₃ alkyl, C₁-C₃ haloalkyl, C₃-C₅ cycloalkyl, C₄-C₆ cycloalkylalkyl, C₄-C₆ alkylcycloalkyl or C₃-C₅ halocycloalkyl;
R² is H, C₁-C₆ alkyl, C₃-C₅ cycloalkyl, C₄-C₆ alkylcycloalkyl, C₄-C₆ cycloalkylalkyl or C₄-C₆ halocycloalkylalkyl;
R³ is C₁-C₁₂ alkyl, —CH₂(C₁-C₁₁ haloalkyl), —CH₂(C₁-C₁₁ alkoxyalkyl), C₃-C₇ cycloalkyl, C₃-C₇ halocycloalkyl or C₄-C₇ cycloalkylalkyl, each optionally substituted with one or more substituents selected from the group consisting of CN, C₁-C₆ alkyl, C₁-C₆ haloalkyl, C₃-C₈ cycloalkyl, C₃-C₈ halocycloalkyl or G¹;
each X is independently halogen, cyano, nitro, C₁-C₆ alkyl, C₃-C₈ cycloalkyl, C₄-C₁₀ alkylcycloalkyl, C₄-C₁₀ cycloalkylalkyl, C₅-C₁₂ alkylcycloalkylalkyl, C₂-C₇ cyanoalkyl, C₂-C₈ alkoxyhaloalkyl, C₁-C₆ alkoxy, C₃-C₈ cycloalkoxy, C₄-C₁₀ cycloalkylalkoxy, C₁-C₆ alkylthio, C₃-C₈ cycloalkylthio, C₁-C₆ alkylsulfinyl, C₁-C₆ alkylsulfonyl, C₃-C₈ cycloalkylsulfonyl, C₃-C₈ cycloalkylsulfinyl, C₂-C₈ dialkylaminosulfonyl, C₂-C₈ alkoxycarbonyl, C₂-C₈ dialkylamino, C₄-C₁₂ dialkylimido, C₃-C₁₀ trialkylsilyl or G²; or
a pair of X substituents attached to contiguous carbon atoms are taken together with said carbon atoms to form a fused phenyl ring, a fused 5- to 6-membered nonaromatic carbocyclic ring, a fused 5- or 6-membered heteroaromatic ring or a fused 5- to 6-membered nonaromatic heterocyclic ring, each fused ring optionally substituted with up to 4 substituents independently selected from R⁴ on carbon ring members and R⁵ on nitrogen ring members;
each G¹ and G² is independently phenyl, benzyl, phenoxy, phenylsulfonyl, pyridinylmethyl, a 3- to 7-membered nonaromatic carbocyclic or heterocyclic ring, a 5- to 6-membered heteroaromatic ring or an 8- to 10-membered aromatic carbobicyclic or heterobicyclic ring system, each optionally substituted with up to 4 substituents independently selected from R⁴ on carbon ring members and R⁵ on nitrogen ring members
each R⁴ is halogen, cyano, nitro, C₁-C₆ alkyl, C₁-C₆ haloalkyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₂ alkylcycloalkylalkyl, C₂-C₈ alkoxyalkyl, C₂-C₈ haloalkoxyalkyl, C₂-C₈ alkoxyhaloalkyl, C₂-C₈ haloalkoxyhaloalkyl, C₄-C₁₀ cycloalkoxyalkyl, C₄-C₁₀ halocycloalkoxyalkyl, C₃-C₁₀ alkoxyalkoxyalkyl, C₁-C₆ alkoxy, C₁-C₆ haloalkoxy, C₃-C₈ cycloalkoxy, C₃-C₈ halocycloalkoxy, C₄-C₁₀ cycloalkylalkoxy, C₂-C₈ alkoxyalkoxy, C₂-C₈ haloalkoxyalkoxy, C₂-C₈ alkoxyhaloalkoxy, C₂-C₈ haloalkoxyhaloalkoxy, C₃-C₁₀ alkoxycarbonylalkoxy, C₂-C₈ alkylthioalkyl, C₁-C₆ alkylthio, C₁-C₆ haloalkylthio, C₃-C₈ cycloalkylthio, C₂-C₈ (alkylcarbonyl)thio, C₂-C₈ (alkylthio)carbonyl, C₂-C₈ alkoxycarbonyl, C₄-C₁₀ cycloalkoxycarbonyl, C₅-C₁₂ cycloalkylalkoxycarbonyl, C₃-C₁₀ dialkylaminocarbonyl, C₂-C₈ alkylsulfinylalkyl, C₂-C₈ alkylsulfonylalkyl, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₈ cycloalkylsulfinyl, C₂-C₈ dialkylaminosulfonyl, C₃-C₁₀ dialkylaminoalkyl, C₂-C₈ dialkylamino, C₃-C₁₀ dialkylamidino, C₂-C₇ cyanoalkyl, C₃-C₁₀ trialkylsilyl, C₃-C₁₀ halotrialkylsilyl, phenyl, pyridinyl, thienyl, naphthalenyl, phenoxy, phenylsulfonyl, pyridinylmethyl or benzyl;
each R⁵ is C₁-C₆ alkyl, C₁-C₆ haloalkyl, C₃-C₈ cycloalkyl, C₃-C₈ halocycloalkyl, C₄-C₁₀ cycloalkylalkyl, C₄-C₁₀ halocycloalkylalkyl, C₄-C₁₀ alkylcycloalkyl, C₅-C₁₂ alkylcycloalkylalkyl, C₆-C₁₄ cycloalkylcycloalkyl, C₄-C₁₀ cycloalkoxyalkyl, C₄-C₁₀ halocycloalkoxyalkyl, C₂-C₈ alkoxyalkyl, C₂-C₈ haloalkoxyalkyl, C₃-C₁₀ alkoxyalkoxyalkyl, C₂-C₈ alkoxycarbonyl, C₄-C₁₀ cycloalkoxycarbonyl, C₅-C₁₂ cycloalkylalkoxycarbonyl, C₃-C₁₀ dialkylaminocarbonyl, C₂-C₈ (alkylthio)carbonyl, C₁-C₆ alkylthio, C₁-C₆ haloalkylthio, C₃-C₈ cycloalkylthio, C₂-C₈ alkylthioalkyl, C₂-C₈ alkylsulfinylalkyl, C₂-C₈ alkylsulfonylalkyl, C₁-C₆ haloalkylsulfonyl, C₃-C₈ cycloalkylsulfonyl, C₂-C₈ dialkylaminosulfonyl, C₄-C₁₀ dialkylaminoalkyl, C₂-C₈ dialkylamino, C₃-C₁₀ trialkylsilyl, C₃-C₁₀ halotrialkylsilyl, C₂-C₇ cyanoalkyl, phenyl, pyridinyl, thienyl, naphthalenyl, phenoxy, phenylsulfonyl, pyridinylmethyl or benzyl; and
m is 0, 1, 2, 3, 4 or 5.

2. A compound of claim 1 wherein:
R¹ is H, C₁-C₃ alkyl, C₁-C₃ haloalkyl or C₃-C₅ cycloalkyl;
R² is H, C₁-C₆ alkyl, C₃-C₅ cycloalkyl or C₄-C₆ cycloalkylalkyl;
R³ is C₁-C₁₂ alkyl, —CH₂(C₁-C₆ haloalkyl), —CH₂(C₁-C₆ alkoxyalkyl), C₃-C₇ cycloalkyl, C₃-C₇ halocycloalkyl, C₃-C₇ cycloalkylalkyl or G¹;
each X is independently halogen, cyano, nitro, C₁-C₆ alkyl, C₃-C₈ cycloalkyl, C₄-C₁₀ alkylcycloalkyl, C₄-C₁₀ cycloalkylalkyl, C₅-C₁₂ alkylcycloalkylalkyl, C₂-C₇ cyanoalkyl, C₂-C₈ alkoxyhaloalkyl, C₁-C₆ alkoxy, C₃-C₈ cycloalkoxy, C₄-C₁₀ cycloalkylalkoxy, C₁-C₆ alkylthio, C₃-C₈ cycloalkylthio, C₁-C₆ alkylsulfinyl, C₁-C₆ alkylsulfonyl, C₃-C₈ cycloalkylsulfonyl, C₃-C₈ cycloalkylsulfinyl, C₂-C₈ dialkylamino, C₄-C₁₂ dialkylimido, C₃-C₁₀ trialkylsilyl or G²; or
a pair of X substituents attached to contiguous carbon atoms are taken together with said carbon atoms to form a fused phenyl ring or 5- to 6-membered nonaromatic carbocyclic ring optionally substituted with up to 4 substituents independently selected from R⁴ on carbon ring members;
each G¹ and G² is independently phenyl, benzyl, phenoxy, naphthalenyl or a 25 5- or 6-membered heteroaromatic ring, each optionally substituted with up to 4 substituents independently selected from R⁴ on carbon ring members and R⁵ on nitrogen ring members;
each R⁴ is halogen, cyano, nitro, C₁-C₆ alkyl, C₁-C₆ haloalkyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₂ alkylcycloalkylalkyl, C₂-C₈ alkoxyalkyl, C₂-C₈ haloalkoxyalkyl, C₂-C₈ alkoxyhaloalkyl, C₂-C₈ haloalkoxyhaloalkyl, C₄-C₁₀ cycloalkoxyalkyl, C₄-C₁₀ halocycloalkoxyalkyl, C₃-C₁₀ alkoxyalkoxyalkyl, C₁-C₆ alkoxy, C₁-C₆ haloalkoxy, C₃-C₈ cycloalkoxy, C₃-C₈ halocycloalkoxy, C₄-C₁₀ cycloalkylalkoxy, C₂-C₈ alkoxyalkoxy, C₂-C₈ haloalkoxyalkoxy, C₂-C₈ alkoxyhaloalkoxy, C₂-C₈ haloalkoxyhaloalkoxy, C₃-C₁₀ alkoxycarbonylalkoxy, C₂-C₈ alkylthioalkyl, C₁-C₆ alkylthio, C₁-C₆ haloalkylthio, C₂-C₈ alkoxycarbonyl, C₄-C₁₀ cycloalkoxycarbonyl, C₅-C₁₂ cycloalkylalkoxycarbonyl, C₂-C₈ alkylsulfonylalkyl, C₁-C₆ alkylsulfinyl, C₁-C₆ haloalkylsulfinyl, C₁-C₆ alkylsulfonyl, C₁-C₆ haloalkylsulfonyl, C₃-C₈ cycloalkylsulfonyl, C₃-C₁₀ dialkylamidino, C₂-C₇ cyanoalkyl, phenyl, pyridinyl, thienyl, naphthalenyl, phenoxy or benzyl;
each R⁵ is C₁-C₆ alkyl, C₁-C₆ haloalkyl, C₃-C₈ cycloalkyl, C₃-C₈ alkoxyalkyl, phenyl or pyridinyl; and
m is 0, 1, 2, or 3.

3. A compound of claim 1 wherein
R¹ is H, C₁-C₃ alkyl or C₁-C₃ haloalkyl;
R² is H or C₁-C₆ alkyl;
R³ is C₁-C₆ alkyl;
each X is independently halogen, cyano, nitro, C₁-C₆ alkyl, C₃-C₈ cycloalkyl or G²; or
a pair of X substituents attached to contiguous carbon atoms are taken together with said carbon atoms to form a fused phenyl ring optionally substituted with up to 4 substituents independently selected from R⁴ on carbon ring members;
each G² is independently phenyl or a 5- or 6-membered heteroaromatic ring, each optionally substituted with up to 4 substituents independently selected from R⁴ on carbon ring members and R⁵ on nitrogen ring members;
R⁴ is halogen, C₁-C₆ alkyl, C₁-C₆ haloalkyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₆-C₁₄ cycloalkylcycloalkyl, C₅-C₁₂ alkylcycloalkylalkyl, C₂-C₈ alkoxyalkyl, C₄-C₁₀ cycloalkoxyalkyl, C₁-C₆ alkoxy, C₁-C₆ haloalkoxy, C₃-C₈ cycloalkoxy, C₄-C₁₀ cycloalkylalkoxy, C₂-C₈ haloalkoxyalkoxy, C₂-C₈ alkoxyhaloalkoxy, C₃-C₁₀ alkoxycarbonylalkoxy, C₁-C₆ alkylthio, C₁-C₆ haloalkylthio, C₂-C₈ alkoxycarbonyl, C₄-C₁₀ cycloalkoxycarbonyl, C₅-C₁₂ cycloalkylalkoxycarbonyl, C₂-C₈ alkylsulfonylalkyl, C₁-C₆ haloalkylsulfonyl, phenyl, phenoxy or benzyl;
each R⁵ is independently C₁-C₆ alkyl or C₁-C₆ haloalkyl; and
m is 1, 2 or 3.

4. A compound of claim 3 wherein
A, B and D are CH,
R¹ is H, CH₃ or CF₃;
R² is H;
R³ is CH₃;
each X is halogen or G²; or
a pair of X substituents attached to contiguous carbon atoms are taken together with said carbon atoms to form a fused phenyl ring, and said X substituents are attached at the 3- and 4-positions or 4- and 5-positions;
each G² is phenyl or pyrazole, each optionally substituted with up to 4 substituents independently selected from R⁴ on carbon ring members and R⁵ on nitrogen ring members;
each R⁴ is halogen, C₁-C₆ alkyl, C₁-C₆ haloalkyl, C₁-C₆ alkoxy, C₁-C₆ haloalkoxy, C₁-C₆ alkylthio, phenyl, phenoxy or benzyl;
m is 1 or 2; and
R⁵ is Me.

5. A compound of claim 4 wherein
each G² is pyrazole attached at the 4-positon of Formula 1 and through the 1-position of the pyrazole ring and optionally substituted with up to 4 substituents independently selected from R⁴ on carbon ring members and R⁵ on nitrogen ring members.

6. A compound of claim 3 wherein
A and B are CH, and D is N
R¹ is H, CH₃ or CF₃;
R² is H;
R³ is CH₃;
each X is halogen or G²; or
two X attached to contiguous carbon atoms are taken together with said carbon atoms to form a fused phenyl ring, and said two X are attached at the 3- and 4-positions or 4- and 5-positions;
each G² is phenyl or pyrazole, each optionally substituted with up to 4 substituents independently selected from R⁴ on carbon ring members and R⁵ on nitrogen ring members;
each R⁴ is halogen, C₁-C₆ alkyl, C₁-C₆ haloalkyl, C₁-C₆ alkoxy, C₁-C₆ haloalkoxy, C₁-C₆ alkylthio, phenyl, phenoxy or benzyl;
m is 1 or 2; and
R⁵ is Me.

7. A compound of claim 1 which is selected from the group consisting of:
rel-3-[(3R,5S)-2,5-dimethyl-3-[4-[3-(trifluoromethyl)-1H-pyrazol-1-yl]phenyl]-5-isoxazolidinyl]pyridine,
rel-3-[(3R,5S)-3-[4-(4-bromo-1H-pyrazol-1-yl)phenyl]-2,5-dimethyl-5-isoxazolidinyl]pyridine,
rel-3-[(3R,5S)-3-(4-iodophenyl)-2-methyl-5-(trifluoromethyl)-5-isoxazolidinyl]pyridine,
rel-3-[(3R,5S)-2,5-dimethyl-3-(4′-methyl[1,1 ′-biphenyl]-4-yl)-5-isoxazolidinyl]pyridine and
rel-2-[(3R,5S)-3-[1,1′-biphenyl]-4-yl-2,5-dimethyl-5-isoxazolidinyl]pyrazine.

8. A fungicidal composition comprising (a) a compound of claim 1; and (b) at least one other fungicide.

9. A fungicidal composition comprising (a) a fungicidally effective amount of at least one compound of claim 1; and (b) at least one additional component selected from the group consisting of surfactants, solid diluents and liquid diluents.

10. A method for controlling plant diseases caused by fungal plant pathogens comprising applying to the plant or portion thereof, or to the plant seed, a fungicidally effective amount of a compound of claim 1.

11. A compound of claim 1 which is: [chemical structure]
